(19) United States
(12) Patent Application Publication
(10) Pub. No.: US 2006/0035250 A1
(43) Pub. Date: Feb. 16, 2006

(54) Necessary and sufficient reagent sets for chemogenomic analysis

(76) Inventor: Georges Natsoulis, Kensington, CA (US)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(21) Appl. No.: 11/149,612

(22) Filed: Jun. 10, 2005

Related U.S. Application Data
(60) Provisional application No. 60/579,183, filed on Jun. 10, 2004.

(51) Int. Cl. C12Q 1/68; G06F 19/00

(52) U.S. Cl. 435/6; 702/20

(57) ABSTRACT

The present invention discloses methods of data analysis directed to diagnostic development, and in particular the development of signatures for classifying chemogenomic data. The invention provides methods for identifying and functionally characterizing a “necessary” set of information rich variables. The invention also discloses methods for identifying a plurality of “sufficient” classifiers. The necessary set of variables may be incorporated into a single diagnostic device to provide simultaneous confirmation of a classification measurement with a plurality of independent classifiers. In the field of biological diagnostics, the invention may be used to provide a plurality of short lists of genes, referred to as “signatures” that are “sufficient” to carry out specific classification tasks such as predicting the activity and side effects of a compound in vivo.

### CROSS REFERENCE TO RELATED APPLICATIONS

This application claims priority from U.S. Provisional Application No. 60/579,183, filed Jun. 10, 2004, which is hereby incorporated by reference in its entirety.

### FIELD OF THE INVENTION

This invention relates to the field of diagnostic development, and in particular the development of chemogenomic signatures or biomarkers. The invention provides methods for identifying a “necessary” set of information rich variables from which a plurality of “sufficient” classifiers may be derived. In the field of biological diagnostics, the invention may be used to provide short lists of genes, referred to as “gene signatures” that may be used to carry out specific classification tasks such as predicting the activity and side effects of a compound in vivo.

### BACKGROUND OF THE INVENTION

A diagnostic assay typically consists of performing one or more measurements and then assigning a sample to one or more categories based on the results of the measurement(s). Thus, most diagnostic devices are simply two-class classifiers. The classifier can be a function of all or of a subset of the initial variables. The value of that function is calculated for each individual datum. The individual sample is assigned to one or the other class depending on whether the result of the classifier function exceeds a defined threshold.

Desirable attributes of a diagnostic assay include high sensitivity and specificity measured in terms of low false negative and false positive rates and overall accuracy. Because diagnostic assays are often used to assign large number of samples to given categories, the issues of cost per assay and throughput (number of assays per unit time or per worker hour) are of paramount importance.

Usually the development of a diagnostic assay involves the following steps: (1) define the class (i.e., the end point) to diagnose, (e.g., cholestasis, a pathology of the liver); (2) identify one or more variables (i.e., measurements) whose value correlates with the end point (e.g., elevation of bilirubin in the bloodstream as an indication of cholestasis); and (3) develop a specific, accurate, high-throughput and cost-effective device for making the specific measurements needed to predict or determine the endpoint.

Over the past 10 years, a variety of techniques have been developed that are capable of measuring a large number of different biological analytes (i.e., variables) simultaneously but which require relatively little optimization for any of the individual analyte detectors. Perhaps the most successful example is the DNA microarray, which may be used to measure the expression levels of thousands or even tens of thousands of genes simultaneously. Based on well-established hybridization rules, the design of the individual probe sequences on a DNA microarray now may be carried out in silicon and without any specific biological question in mind. Although DNA microarrays have been used primarily for pure research applications, this technology currently is being developed as a medical diagnostic device and everyday bioanalytical tool.

Although DNA microarrays are considerably more expensive than conventional diagnostic assays they do offer two critical advantages. First, they tend to be more sensitive, and therefore more discriminating and accurate in prediction than most current diagnostic techniques. Using a DNA microarray, it is possible to detect a change in a particular gene's expression level earlier, or in response to a milder treatment than is possible with more classical pathology markers. Also, it is possible to discern combinations of genes or proteins useful for resolving subtle differences in forms of an otherwise more generic pathology. Second, because of their massively parallel design, DNA microarrays make it possible to answer many different diagnostic questions. In addition, by using different combinations of variables that may be available on an array, it may be possible to confirm the answer to a single classification question in multiple independent ways and thereby increase accuracy.

A key challenge in developing the DNA microarray as a diagnostic tool lies in accurately interpreting the large amount of multivariate data provided by each measurement (i.e., each probe's hybridization). Indeed, commercially available high density DNA microarrays (also referred to as “gene chips” or “biochips”) allow one to collect thousands of gene expression measurements using standardized published protocols. However, typically only a very small fraction of these measurements are relevant to a given diagnostic classification question being asked by the user. For example, only 10-20 genes (out of 10,000 available on the microarray) may be used as the gene signature for a specific question. Thus, current DNA microarrays provide a large amount of information that is not used for answering most typical diagnostic assay questions. Similar data overload problems exist in adapting other highly multiplexed bioassays such as RT-PCR or proteomic mass spectrometry to diagnostic applications.

A recently developed powerful new application for the DNA microarray is chemogenomic analysis. The term “chemogenomics” refers to the transcriptional and/or bioassay response of one or more genes upon exposure to a particular chemical compound. A comprehensive database of chemogenomic annotations for large numbers of genes in response to large numbers of chemical compounds may be used to design and optimize new pharmaceutical lead compounds based only on a transcriptional and biomolecular profile of the known (or merely hypothetical) compound. For example, a small number of rats may be treated with a novel lead compound and then expression profiles measured for different tissues from the compound treated animals using DNA microarrays. Based on the correlative analysis of this compound treatment expression level data with respect to the chemogenomic reference database, it may be possible to predict the toxicological profile and/or likely off-target effects of the new compound. Construction of a comprehensive chemogenomic database and methods for chemogenomic analysis using microarrays are described in Published U.S. patent application No. 2005/0060102 A1, which is hereby incorporated herein by reference in its entirety.

Systematic “mining” of large chemogenomic datasets has led to the discovery of new relationships between genes. It has also led to new insight into the genes and pathways affected by particular classes of compound treatments. An important tool for discovering these new relationships are specific, short weighted lists of genes that may be used to determine whether certain gene expression changes are related (i.e., whether the observed effects are in the same class). These gene lists, referred to as “gene signatures,” provide simple, robust tools for answering classification questions using DNA microarrays. Methods for deriving and using gene signatures to analyzed chemogenomic data are disclosed in Published U.S. patent application No. 2005/0060102 A1 and PCT Publication No. WO 2004/037200, each of which is hereby incorporated herein by reference in its entirety.

The use of gene signatures to answer diagnostic questions is not limited to the DNA hybridization assay context. The general concept of signatures may be widely applied to any analytical testing situation that may be reduced to a question of whether data are within or outside a specific class.

Even with robust gene signatures, however, sometimes data are measured that defy simple classification algorithms. That is, the signature does not clearly place the data in either of the two classes it defines. This may be due to the nature of the data originally used to derive the signature (i.e., the signature is not robust enough) or it may indicate that the data defines a new class. New methods are needed to derive signatures capable of classifying this type of “borderline” data. The availability of improved signatures would greatly increase the usefulness of these signatures as accurate and reliable tools for diagnostic classification.

### SUMMARY OF THE INVENTION

In one embodiment, the present invention provides a method of selecting a set of necessary variables useful for answering a classification question comprising: (a) providing a full multivariate dataset; (b) querying the full dataset with a classification question so as to generate a first linear classifier comprising a first set of variables and capable of performing with a log odds ratio greater than or equal to a selected threshold value (e.g., log odds ratio greater than or equal to 4.0); and (c) removing the first set of variables from the full dataset thereby generating a partially depleted dataset; (d) querying the partially depleted dataset with the classification question so as to generate a second linear classifier comprising a second set of variables; repeating steps c and d until the linear classifier generated is not capable of performing with a log odds ratio greater than or equal to the selected threshold (or second different threshold); and selecting the variables of the linear classifiers meeting the performance threshold; wherein the remaining fully depleted subset of variables is unable to answer the classification question with a log odds ratio greater than the selected threshold. In one preferred embodiment, a single log odds ratio threshold of greater than or equal to 4.0 is used. In an alternative embodiment of the method, a second threshold may be selected and used to determine the performance of the remaining variables when repeating steps c and d. In one embodiment, the method may be carried out wherein the multivariate dataset comprises chemogenomics data, and specifically, comprises a dataset from polynucleotide array experiments on compound-treated samples. In another preferred embodiment of the above method, the linear classifiers are sparse, that is they are composed of short gene lists. In a preferred embodiment, the sparse linear classifiers are generated with an algorithm selected from the group consisting of SPLP, SPLR and SPMPM. In another embodiment the above method is carried out with a multivariate dataset comprising data from a proteomic or metabolomic experiment.

The present invention also includes a set of necessary variables for answering classification questions made according to the method described above. Necessary sets of the invention may be quite large and include all or nearly all variables in the full set of variables. In preferred embodiments, the variables in the necessary sets of the invention are genes and number fewer than 400, 300, 200, 100, or 50 genes In one preferred embodiment, the necessary sets of variables of the present invention number fewer than 4%, 3%, 2%, 1% or 0.5% of the total number of genes present on a typical DNA microarray that includes on the order of 8,000, 10,000 or even 20,000 or more genes.

The present invention also includes an array, or other diagnostic device, comprising a set of polynucleotides each representing a gene in the necessary set made according to the method described above.

In another embodiment, the invention includes a diagnostic reagent set useful in diagnostic assays and diagnostic kits for a specific classification question comprising a set of polynucleotides each representing a gene in the necessary set made according to the above method.

In another embodiment, the invention includes a subset of genes useful for answering a chemogenomic classification question (including those questions disclosed in Table 2) comprising a percentage of genes randomly selected from necessary set made according to method described above, wherein the addition of the percentage of genes to the depleted set for the classification question increases the average logodds ratio of the linear classifiers generated by the depleted set. In some embodiments, the subset may be defined according to the percentage increase in the average LOR performance of the depleted set, in other embodiments, the increase corresponds to a set average LOR threshold.

In one specific embodiment, the subset of genes is useful for answering the monoamine re-uptake (SERT) inhibitor classification question and the necessary set consists of the 311 genes listed in Table 5. In one preferred embodiment, the subset comprises a randomly selected 15% of genes from the 311 in the SERT necessary set and the average logodds ratio is increased to greater than or equal to 3.0. In another preferred embodiment, the subset comprises a randomly selected 26% of genes from the 311 in the SERT necessary set and the average logodds ratio is increased to greater than or equal to 4.0.

In another embodiment, the invention includes a diagnostic assay comprising a set of secreted proteins encoded by the genes of a necessary set made according to the above-described method (e.g., an array of immobilized receptors), or an assay comprising reagents capable of detecting secreted proteins encoded by the genes of a necessary set.

In another embodiment, the invention provides a method for preparing a reagent set comprising the steps of: (a) deriving a first linear classifier comprising a first set of genes from a full dataset, wherein said first linear classifier is capable of answering a classification question with a log odds ratio greater than or equal to a first selected threshold value; (b) removing said first set of genes from the full dataset thereby resulting in a partially depleted chemogenomic dataset; (c) deriving a second linear classifier comprising a second set of genes from the partially depleted dataset, wherein the second linear classifier capable of answering a classification question with a log odds ratio greater than or equal to a second selected threshold value; (d) removing said second set of genes from the partially depleted dataset; (e) preparing a plurality of isolated polynucleotides or polypeptides, wherein each polynucleotide or polypeptide is capable of detecting at least one gene of said first and second sets genes. This method of preparing a reagent set may further include the steps of: after step (d) repeating the steps of (i) deriving a linear classifier; and (ii) removing each additional linear classifier's set of genes from the partially depleted dataset; until the partially depleted dataset is not capable of generating a linear classifier with a log odds ratio greater than or equal to the second selected threshold value.

In another embodiment, the invention provides a reagent set for analysis of a chemogenomic classification question comprising a set of polynucleotides or polypeptides representing a plurality of genes, wherein a random selection of at least 10% of said plurality of genes restores the ability of a depleted set to generate signatures for the classification question with an average LOR greater than or equal to 4.0, wherein the depleted set cannot generate a signature with an average LOR of greater than 1.2,. In other embodiments, the reagent set represents a plurality of genes, wherein the random selection capable of restoring the ability of the depleted set is of at least 15%, 20%, 25%, 30%, 35%, 40%, 45%, 50%, 55%, 60%, 65%, 70%, 75% or 80% of said plurality of genes. In other embodiments, the reagent set represents a plurality of genes, whether a random selection of at least 10% of said plurality of genes restores the ability of a depleted set to generate signatures for the classification question with an average LOR greater than or equal to 3.0, 4.0, 5.0, 6.0, 7.0, or 8.0. In one embodiment, the reagent set comprises polypeptides represent genes capable of detected secreted proteins.

In another embodiment, the invention provides a set of necessary variables for answering a classification question comprising the variables whose removal from a full multivariate dataset results in a depleted set of variables that are unable to answer the classification question with a performance greater than some selected threshold (e.g., log odds ratio greater than or equal to 4.0). In preferred embodiments, the variables in the necessary sets of the invention are genes and number fewer than 400, 300, 200, 100, 50 or even 25 genes. In one preferred embodiment, the necessary sets of variables of the present invention are genes and number fewer than 4%, 3%, 2%, 1% or 0.5% of the total number of genes present in a complete set of 8,000, 10,000 or even 20,000 or more genes.

In another embodiment, the invention includes a diagnostic device (e.g., an array), a diagnostic reagent set, or a diagnostic kit, useful for answering a classification question, comprising a set of polynucleotides representing a plurality of genes, wherein removal of the plurality of genes from a full DNA array dataset results in a depleted set of genes that is unable to generate signatures for the classification question with an average log odds ratio greater than or equal to a chosen threshold. In other embodiments, the chosen threshold is an average LOR greater than or equal to 3.0, 4.0, 5.0, 6.0, 7.0, or 8.0.

In an alternative embodiment, the invention provides a diagnostic device comprising a set of secreted proteins encoded by the genes in the necessary set for a specific classification question or a set of reagents capable of detecting said secreted proteins.

In one embodiment, the present invention provides a method of identifying non-overlapping sufficient sets of variables useful for answering a classification question comprising: providing a full multivariate dataset; querying the full dataset with a classification question so as to generate a first linear classifier capable of performing with a log odds ratio greater than or equal to a chosen threshold and comprising a first set of variables; removing the first set of variables from the full dataset thereby generating a partially depleted dataset; and querying the partially depleted dataset with the classification question so as to generate a second linear classifier capable of performing with a log odds ratio greater than or equal to a chosen threshold and comprising a second set of variables; wherein none of the variables in the second set overlaps the variables in the first set.

In one embodiment, the method of identifying non-overlapping sufficient sets may be carried out wherein the multivariate dataset comprises chemogenomics data, and specifically, comprises a dataset from polynucleotide array experiments on compound-treated samples. In another preferred embodiment of the above method, the linear classifiers are reducible to weighted gene lists. In another embodiment the above method is carried out with a multivariate dataset comprising data from a proteomic experiment.

The present invention also provides a method of classifying experimental data comprising: providing at least two non-overlapping sufficient sets of variables useful for answering a classification question; querying the experimental data with one of the at least two non-overlapping sufficient sets of variables; querying the experimental data with another of the at least two non-overlapping sufficient sets of variables; wherein the classification of the data is determined based on the answers to the queries generated by the at least two non-overlapping sets of variables.

### BRIEF DESCRIPTION OF THE DRAWINGS

FIG. 1 depicts a schematic representation of a multivariate dataset and the relationship between the subsets of variables capable of answering a specific classification question, i.e., the necessary and sufficient sets of variables (e.g., genes) produced according to the methods of the present invention.

FIGS. 2(A) and (B) depict results of repeatedly applying the stripping algorithm for four different classification questions used to query a chemogenomic dataset. Four signatures were chosen. One of them, used here as a control (NSAID, Cox2/1, coxib-like) failed at the 2^{nd} cycle in the previous analysis (Classification #39 in Table 3). (A) shows the evolution of the Test Log Odds Ratio as function of the cycles of stripping. (B) shows the cumulative number of genes used.

FIG. 3 depicts results of the analysis of a monoamine reuptake inhibitors (SERT) signature. The initial SERT signature (Classification #1 in Table 3) is 79 genes long and its performance is LOR=5.92. Specifically, 5, 10, 20, 40, 80% subset of genes chosen randomly either from the necessary set of 311 genes (circles) or the random set of 311 genes (crosses) were added to the 7943 gene set. This process was repeated 50 times. The table presents the mean and standard deviations of the LOR for each subset size added to the depleted set. The plot shows the distribution of the LOR (test LOR obtained for a single 60/40 partition of the dataset in each case) obtained when each of these genes lists is used as input to recompute the same SERT signature. An interpolation of the LOR=4.0 crossing point (indicated by arrow) shows that a randomly chosen 26% of the necessary set can restore an average performance of LOR=4.0.

FIG. 4 depicts a clustered table of impact values for the 317 genes (y-axis) that appear in the first 5 cycles of stripping of the PPARα signature versus all 1441 compound treatments whose gene expression was measure in rat liver tissue (x-axis). The table was clustered using the UPGMA algorithm available in the Spotfire Decision Site™ software package. Impact was defined as the product of a gene's weight by the log ratio of expression in a given treatment. Negative impact values are colored green and positive are colored red. At the extreme right a “total impact” column was added. This column represents the sum of the impact values for a gene across all treatments. Strong positive values are in red, all other values are green.

FIG. 5 depicts results confirming that compounds are signature hits. The left panel shows the maximum scalar product achieved by a given compound against any of the first 5 PPARα signatures, as defined above. The complete table encompasses 329 compounds. The label of each compound is shown next to the compound name. Seven compounds are part of the class of interest (PPARα) and labeled “+1”. The unknown compound is labeled as “0” and ten randomly chosen non-PPARα compounds are given a label “−2”. These are not part of the signature generation. The signature is training against all other (˜300) non-PPARα compounds labeled as “−1” and not shown in the table. The same data is expressed as a rank in the right panel.

FIG. 6 depicts plot of GO terms identified at different stripping cycles during the generation of the PPARα necessary set.

FIG. 7 depicts plot of GO terms identified at different stripping cycles during the generation of the HMGcoA-statin necessary set.

### DETAILED DESCRIPTION OF THE INVENTION

### I. OVERVIEW

The present invention provides a method of defining a “necessary” set of variables from which multiple independent classifiers (e.g., gene signatures) may be derived. Using multiple independent signatures for the same classification question in a single classification experiment (e.g., in a single microarray assay) it is possible to analyze “borderline” data more accurately. For example, two non-overlapping gene signatures that classify a specific type of pathway inhibitors may be used to reach a consensus classification for a particular compound that does not score highly with either signature alone.

In addition, the necessary set itself, which may be derived for any classification question according to the methods disclosed herein, represents a source of information rich variables that may be used to prepare diagnostic devices. As shown herein, even a small percentage of genes randomly selected from the necessary set for a specific classification question may be used to “revive” a depleted dataset.

In addition to providing an improved diagnostic tool, the comparative analysis of the multiple independent and/or non-overlapping signatures that exist within a “necessary” set of variables, can provide insight into structural and functional features of the full dataset from which the signatures are derived. For example, by using a method of sequentially “stripping” away gene signatures from the full dataset to reveal underlying gene signatures associated with distinct metabolic pathways. These distinct and independent signatures can provide an alternative signature useful for development of a novel diagnostic test. Thus, the present invention provides tools to develop novel toxicology or pharmacology signatures, or diagnostic assays.

### II. DEFINITIONS

“Multivariate dataset” as used herein, refers to any dataset comprising a plurality of different variables including but not limited to chemogenomic datasets comprising logratios from differential gene expression experiments, such as those carried out on polynucleotide microarrays, or multiple protein binding affinities measured using a protein chip. Other examples of multivariate data include assemblies of data from a plurality of standard toxicological or pharmacological assays (e.g., blood analytes measured using enzymatic assays, antibody based ELISA or other detection techniques).

“Variable” as used herein, refers to any value that may vary. For example, variables may include relative or absolute amounts of biological molecules, such as mRNA or proteins, or other biological metabolites. Variables may also include dosing amounts of test compounds.

“Classifier” as used herein, refers to a function of a set of variables that is capable of answering a classification question. A “classification question” may be of any type susceptible to yielding a yes or no answer (e.g., “Is the unknown a member of the class or does it belong with everything else outside the class?”). “Linear classifiers” refers to classifiers comprising a first order function of a set of variables, for example, a summation of a weighted set of gene expression logratios. A valid classifier is defined as a classifier capable of achieving a performance for its classification task at or above a selected threshold value. For example, a log odds ratio ≧4.00 represents a preferred threshold of the present invention. Higher or lower threshold values may be selected depending of the specific classification task.

“Signature” as used herein, refers to a combination of variables, weighting factors, and other constants that provides a unique value or function capable of answering a classification question. A signature may include as few as one variable. Signatures include but are not limited to linear classifiers comprising sums of the product of gene expression logratios by weighting factors and a bias term.

“Weighting factor”(or “weight”) as used herein, refers to a value used by an algorithm in combination with a variable in order to adjust the contribution of the variable.

“Impact factor” or “Impact” as used herein in the context of classifiers or signatures refers to the product of the weighting factor by the average value of the variable of interest. For example, where gene expression logratios are the variables, the product of the gene's weighting factor and the gene's measured expression log₁₀ ratio yields the gene's impact. The sum of the impacts of all of the variables (e.g., genes) in a set yields the “total impact” for that set.

“Scalar product”(or “Signature score”) as used herein refers to the sum of impacts for all genes in a signature less the bias for that signature. A positive scalar product for a sample indicates that it is positive for (i.e., a member of) the classification that is determined by the classifier or signature.

“Sufficient set” as used herein is a set of variables (e.g., genes, weights, bias factors) whose cross-validated performance for answering a specific classification question is greater than an arbitrary threshold (e.g., a log odds ratio ≧4.0).

“Necessary set” as used herein is a set of variables whose removal from the full set of all variables results in a depleted set whose performance for answering a specific classification question does not rise above an arbitrarily defined minimum level (e.g., log odds ratio ≧4.00).

“Log odds ratio” or “LOR” is used herein to summarize the performance of classifiers or signatures. LOR is defined generally as the natural log of the ratio of the odds of predicting a subject to be positive when it is positive, versus the odds of predicting a subject to be positive when it is negative. LOR is estimated herein using a set of training or test cross-validation partitions according to the following equation,

$$LOR = ln \frac{( {\sum }_{i = 1}^{c} {TP}_{i} + 0.5 ) * ( {\sum }_{i = 1}^{c} {TN}_{i} + 0.5 )}{( {\sum }_{i = 1}^{c} {FP}_{i} + 0.5 ) * ( {\sum }_{i = 1}^{c} {FN}_{i} + 0.5 )}$$

where c (typically c=40 as described herein) equals the number of partitions, and TPᵢ, TNᵢ, FPᵢ, and FNᵢ represent the number of true positive, true negative, false positive, and false negative occurrences in the test cases of the i^{th} partition, respectively.

“Array” as used herein, refers to a set of different biological molecules (e.g., polynucleotides, peptides, carbohydrates, etc.). An array may be immobilized in or on one or more solid substrates (e.g., glass slides, beads, or gels) or may be a collection of different molecules in solution (e.g., a set of PCR primers). An array may include a plurality of biological polymers of a single class (e.g., polynucleotides) or a mixture of different classes of biopolymers (e.g., an array including both proteins and nucleic acids immobilized on a single substrate).

“Array data” as used herein refers to any set of constants and/or variables that may be observed, measured or otherwise derived from an experiment using an array, including but not limited to: fluorescence (or other signaling moiety) intensity ratios, binding affinities, hybridization stringency, temperature, buffer concentrations.

“Proteomic data” as used herein refers to any set of constants and/or variables that may be observed, measured or otherwise derived from an experiment involving a plurality of mRNA translation products (e.g., proteins, peptides, etc) and/or small molecular weight metabolites or exhaled gases associated with these translation products.

### III. METHODS OF THE INVENTION

Sparse linear classifiers may be used to classify large multivariate datasets from DNA microarray experiments. Sparse as used here means that the vast majority of the variables have zero weight. Sparsity ensures that the sufficient and necessary gene lists produced by the methodology described above are as short as possible. The output is a short weighted gene list (i.e., a gene signature) capable of assigning an unknown treatment to one of two classes. The sparsity and linearity of the classifiers are important features. The linearity of the classifier facilitates the interpretation of the signature—the contribution of each gene to the classifier corresponds to the product of its weight and the value (i.e., logratio) from the microarray experiment. The property of sparsity ensures that the classifier uses only a few genes, which also helps in the interpretation. More importantly, however, because of sparsity the classifier may be reduced to a practical diagnostic device comprising a relatively small set of genes.

A linear classifier generated according to this invention is “sufficient” to classify. In fact, it may be the best list derivable by the algorithm for the task. Significantly, it may be possible to define other gene lists, possibly not overlapping with the first list that can classify the same data. Those other lists likely exhibit a lower performance than the initial list but may still perform better than a given threshold of performance.

The invention provides a method to derive multiple non-overlapping gene signatures for a given question. Because these non-overlapping signatures use different genes they may be used to provide an independent confirmation of the class assignment of an individual sample. Consequently, this method is useful to confirm that an unknown is a member of a given class or to confirm that a known individual is not a member of a class.

The present invention provides a method to identify all of the genes “necessary” to create a classifier that performs above a certain minimal threshold level for a specific classification question. The method also leads to a separate set of “depleted” genes which cannot be used to create a valid linear classifier for a given question.

A. Multivariate Datasets

a. Various Useful Multivariate Data Types

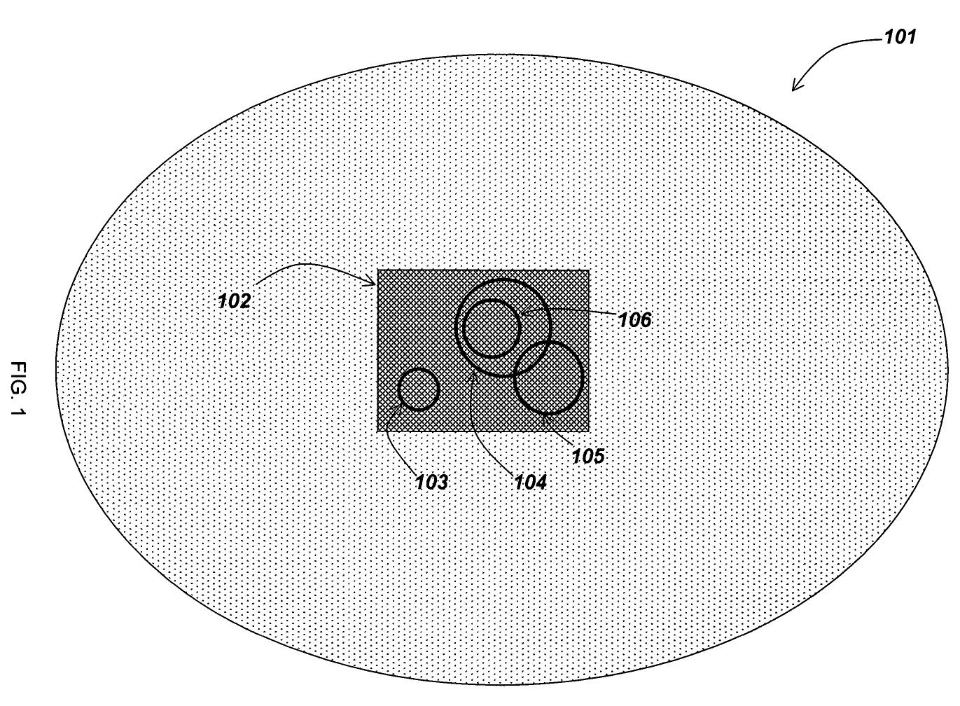

The present invention may be used with a wide range of multivariate data types to identify necessary and sufficient sets of variables useful for generating linear classifiers. FIG. 1 depicts a schematic representation of a multivariate dataset and the resulting subsets of variables capable of answering a specific classification question, i.e., the necessary and sufficient sets of variables produced according to the teachings of the present invention. The largest oval (101) represents the full multivariate dataset. The darker shaded box within the full dataset (102) represents the “necessary” set of variables for a specific classifier. In one method of the present invention, this members of the necessary set may be determined by using a “stripping” algorithm on the full dataset. Accordingly, the variables in the full dataset (101) that are not encompassed within the box (102) form the “depleted” set that is not capable of answering the specific classification question with a defined level of performance. That is, repeated attempts to query the depleted set with the classification question and generate a valid classifier will result in classifiers with a mean performance below the threshold for validity used in stripping the full dataset. Although not explicitly depicted in the figure, it is understood that “partially depleted” sets also exist where only some but not all of the variables in the necessary set have been stripped from the full dataset.

The smaller circles (103-106) inside the necessary set box depicted in FIG. 1 represent the various “sufficient” sets of variables. Each of these sufficient sets is capable of answering the specific classification question with a level of performance above the defined threshold for a valid classifier. The schematic of FIG. 1 illustrates that a plurality of different sized sufficient sets of variables may be generated all of which are encompassed within the necessary set. Further, as shown by circles 104 and 106, some sufficient sets of variables capable of answering a classification question may be entirely contained within others, while others may partially overlap (e.g., circles 104 and 105), or not overlap at all (e.g., circle 103). As discussed below, the classifiers consisting of the variables from two or more non-overlapping sufficient sets may be used together to provide independent confirmation of the answer to a classification question.

A preferred embodiment is the application of the present invention with data generated by high-throughput biological assays such as DNA array experiments, or proteomic assays. For example, as larger multivariate data sets are assembled for large sets of molecules (e.g., small or large chemical compounds) the present method may be applied to these datasets to allow facile generation of multiple, non-overlapping linear classifiers. The large datasets may include any sort of molecular characterization information including, e.g., spectroscopic data (e.g., UV-Vis, NMR, IR, mass spectrometry, etc.), structural data (e.g., three-dimensional coordinates) and functional data (e.g., activity assays, binding assays). The classifiers produced by using the present invention with such a dataset be applied in a multitude of analytical contexts, including the development and manufacture of derivative detection devices (i.e., diagnostics). For example, one may use the present invention with a large multivariate dataset of human metabolite levels to generate classifiers useful in a simplified device for detecting various different ingested toxins used by emergency medical personnel.

Generally, the present invention will be useful wherever it is necessary to simplify data classification. One of ordinary skill will recognize that the methods of the present invention may be applied to multivariate data in areas outside of biotechnology, chemistry, pharmaceutical or the life sciences. For example, the present invention may be used in physical science applications such as climate prediction, or oceanography, where it is essential to prepare simple signatures capable of being used for detection.

Large dataset classification problems are common in the finance industry (e.g., banks, insurance companies, stock brokers, etc.) A typical finance industry classification question is whether to grant a new insurance policy (or home mortgage) versus not. The variables to consider are any information available on the prospective customer or, in the case of stock, any information on the specific company or even the general state of the market. The finance industry equivalent to the “gene signatures” described in the Examples below would be financial signatures for a specific financing decision. The present invention would identify a necessary set of financial variables useful for generating financial signatures capable of answering a specific financing question.

b. Construction of a Multivariate Dataset

As discussed above, the method of the present invention may be used to identify necessary and sufficient subsets of responsive variables within any multivariate data set that are useful for answering classification questions. In preferred embodiments the multivariate dataset comprises chemogenomic data. For example, the data may correspond to treatments of organisms (e.g., cells, worms, frogs, mice, rats, primates, or humans etc.) with chemical compounds at varying dosages and times followed by gene expression profiling of the organism's transcriptome (e.g., measuring mRNA levels) or proteome (e.g., measuring protein levels). In the case of multicellular organisms (e.g., mammals) the expression profiling may be carried out on various tissues of interest (e.g., liver, kidney, marrow, spleen, heart, brain, intestine). Typically, valid sufficient classifiers or signatures may be generated that answer questions relevant to classifying treatments in a single tissue type. The present specification describes examples of necessary and sufficient sets of genes useful for classifying chemogenomic data in liver tissue. The methods of the present invention may also be used however, to generate signatures in any tissue type. In some embodiments, classifiers or signatures may be useful in more than one tissue type. Indeed, a large chemogenomic dataset, like that exemplified in Example 1 may reveal gene signatures in one tissue type (e.g., liver) that also classify pathologies in other tissues (e.g., intestine).

In addition to the expression profile data, the present invention may be useful with chemogenomic datasets including additional data types such as data from classic biochemistry assays carried out on the organisms and/or tissues of interest. Other data included in a large multivariate dataset may include histopathology, pharmacology assays, and structural data for the chemical compounds of interest. Such a multi-data type database permits a series of correlations to be made across data types, thereby providing insights not possible otherwise. For example, a histopathology may be correlated with an expression pattern which is then correlated with an off-target pathway of a class of compound structures. One example of a chemogenomic multivariate dataset particularly useful with the present invention is a dataset based on DNA array expression profiling data as described in U.S. patent application Ser. No. 09/977,064 filed Oct. 11, 2001 (titled “Interactive Correlation of Compound Information and Genomic Information”), which is hereby incorporated by reference for all purposes. Microarrays are well known in the art and consist of a substrate to which probes that correspond in sequence to genes or gene products (e.g., cDNAs, mRNAs, cRNAs, polypeptides, and fragments thereof), can be specifically hybridized or bound at a known position. The microarray is an array (i.e., a matrix) in which each position represents a discrete binding site for a gene or gene product (e.g., a DNA or protein), and in which binding sites are present for many or all of the genes in an organism's genome.

As disclosed above, a treatment may include but is not limited to the exposure of a biological sample or organism (e.g., a rat) to a drug candidate (or other chemical compound), the introduction of an exogenous gene into a biological sample, the deletion of a gene from the biological sample, or changes in the culture conditions of the biological sample. Responsive to a treatment, a gene corresponding to a microarray site may, to varying degrees, be (a) up-regulated, in which more mRNA corresponding to that gene may be present, (b) down-regulated, in which less mRNA corresponding to that gene may be present, or (c) unchanged. The amount of up-regulation or down-regulation for a particular matrix location is made capable of machine measurement using known methods (e.g., fluorescence intensity measurement). For example, a two-color fluorescence detection scheme is disclosed in U.S. Pat. Nos. 5,474,796 and 5,807,522, both of which are hereby incorporated by reference herein. Single color schemes are also well known in the art, wherein the amount of up- or down-regulation is determined in silico by calculating the ratio of the intensities from the test array divided by those from a control.

After treatment and appropriate processing of the microarray, the photon emissions are scanned into numerical form, and an image of the entire microarray is stored in the form of an image representation such as a color JPEG or TIFF format. The presence and degree of up-regulation or down-regulation of the gene at each microarray site represents, for the perturbation imposed on that site, the relevant output data for that experimental run or scan.

The methods for reducing datasets disclosed herein are broadly applicable to other gene and protein expression data. For example, in addition to microarray data, biological response data including gene expression level data generated from serial analysis of gene expression (SAGE, supra) (Velculescu et al., 1995, Science, 270:484) and related technologies are within the scope of the multivariate data suitable for analysis according to the method of the invention. Other methods of generating biological response signals suitable for the preferred embodiments include, but are not limited to: traditional Northern and Southern blot analysis; antibody studies; chemiluminescence studies based on reporter genes such as luciferase or green fluorescent protein; Lynx; READS (GeneLogic); and methods similar to those disclosed in U.S. Pat. No. 5,569,588 to Ashby et. al., “Methods for drug screening,” the contents of which are hereby incorporated by reference into the present disclosure.

In another preferred embodiment, the large multivariate dataset may include genotyping (e.g., single-nucleotide polymorphism) data. The present invention may be used to generate necessary and sufficient sets of variables capable of classifying genotype information. These signatures would include specific high-impact SNPs that could be used in a genetic diagnostic or pharmacogenomic assay.

The method of generating classifiers from a multivariate dataset according to the present invention may be aided by the use of relational database systems (e.g., in a computing system) for storing and retrieving large amounts of data. The advent of high-speed wide area networks and the internet, together with the client/server based model of relational database management systems, is particularly well-suited for meaningfully analyzing large amounts of multivariate data given the appropriate hardware and software computing tools. Computerized analysis tools are particularly useful in experimental environments involving biological response signals. Generally, multivariate data may be obtained and/or gathered using typical biological response signals. Responses to biological or environmental stimuli may be measured and analyzed in a large-scale fashion through computer-based scanning of the machine-readable signals, e.g., photons or electrical signals, into numerical matrices, and through the storage of the numerical data into relational databases. For example a large chemogenomic dataset may be constructed as described in U.S. patent application Ser. No. 09/977,064 filed Oct. 11, 2001 (titled “Interactive Correlation of Compound Information and Genomic Information”) which is hereby incorporated by reference for all purposes.

B. Generating Valid Classifiers from a Dataset

a. Mining of a Large Multivariate Dataset for Classifiers

Generally classifiers or signatures are generated (i.e., mined) from a large multivariate dataset by first labeling the full dataset according to known classifications and then applying an algorithm to the full dataset that produces a linear classifier for each particular classification question. Each signature so generated is then cross-validated using a standard split sample procedure.

The initial questions used to classify (i.e., the classification questions) a large multivariate dataset may be of any type susceptible to yielding a yes or no answer. The general form of such questions is: “Is the unknown a member of the class or does it belong with everything else outside the class?” For example, in the area of chemogenomic datasets, classification questions may include “mode-of-action” questions such as “All treatments with drugs belonging to a particular structural class versus the rest of the treatments” or pathology questions such as “All treatments resulting in a measurable pathology versus all other treatments.” In the specific case of chemogenomic datasets based on gene expression, it is preferred that the classification questions are further categorized based on the tissue source of the gene expression data. Similarly, it may be helpful to subdivide other types of large data sets so that specific classification questions are limited to particular subsets of data (e.g., data obtained at a certain time or dose of test compound). Typically, the significance of subdividing data within large datasets become apparent upon initial attempts to classify the complete dataset. A principal component analysis of the complete data set may be used to identify the subdivisions in a large dataset (see e.g., US 2003/0180808 A1, published Sep. 25, 2003, which is hereby incorporated by reference herein.) Methods of using classifiers to identify information rich genes in large chemogenomic datasets is also described in U.S. Ser. No. 11/114,998, filed Apr. 25, 2005, which is hereby incorporated by reference herein for all purposes.

Labels are assigned to each individual (e.g., each compound treatment) in the dataset according to a rigorous rule-based system. The +1 label indicates that a treatment falls in the class of interest, while a −1 label indicates that the variable is outside the class. Information used in assigning labels to the various individuals to classify may include annotations from the literature related to the dataset (e.g., known information regarding the compounds used in the treatment), or experimental measurements on the exact same animals (e.g., results of clinical chemistry or histopathology assays performed on the same animal).

As more detailed description of 101 classification questions directed to liver tissue are provided in Table 2 in the Examples section below. The “Classification Name” column lists an abbreviated name or description for the particular classification. “Tissue” indicates the tissue from which the signature was derived. Generally, the gene signature works best for classifying gene expression data from tissue samples from which it was derived. In the present example, all 101 signatures generated are valid in liver tissue. The “Universe Description” is a description of the samples that will be classified by the signature. The chemogenomic dataset described in Example 1 contains information from several tissue types at multiple doses and multiple time points. In order to derive gene signatures it is often useful to restrict classification to only parts of the dataset. So for example, it often is useful to restrict classification to a signature tissue. Other common restrictions are to specific time points, for example day 3 or day 5 time points. The “Universe Description” contains phrases like “Tissue=Liver and Timepoint>=3” which, translates into a restriction that the signature will be derived from compound treatments measured by gene expression analysis of liver tissue on days 3,5 or 7 (or later if available). Other phrases might say, “Not (Activity_Class_Union=***BLANK***)” which translates into a restriction that any treatment for which the compound has not been annotated with an “Activity_Class_Union” be excluded from the Universe definition. “Class+1 Description” lists descriptions of the definition of the compound treatments in the chemogenomic database that were labeled in the positive group for deriving the signature. “Class−1 description” is the description of the compound treatments that were labeled as not in the class for deriving the signature. “Class 0 description” are the compound treatments that were not used to derive the gene signature. The 0 label is used to exclude compounds for which the +1 or −1 label is ambiguous. For example, in the case of a literature pharmacology signature, there are cases where the compound is neither an agonist or an antagonist but rather a partial agonist. In this case, the safe assumption is to derive a gene signature without including the gene expression data for this compound treatment. Then the gene signature may be used to classify the ambiguous compound after it has been derived. “LOR” refers to the average logodds ratio which is a measure of the performance of each signature.

As listed in Table 2, there are several different types of class descriptions used to characterize the classification questions. “Structure Activity Class” (SAC) is a description of both the chemical structure and the pharmacological activity of the compound. Thus, for example, estrogen receptor agonists form one group. Another example: bacterial DNA gyrase inhibitor, 8-fluoro-fluoroquinolone and 8-alkoxy-fluoroquinolone antibiotics each form separate SAC classes even though both share the same pharmacological target, DNA gyrase. “Activity_Class_Union” (also referred to as “Union Class”) is a higher level description of several SAC classes. For example, the DNA gyrase Union Class would include both 8-fluoro-fluoroquinolone and 8-alkoxy-fluoroquinolone antibiotics.

Compound activities are also referred to in the class descriptions listed in Table 2. The exact assay referred to in each activity measurement is encoded as “IC50-XXXXX|Assay name,” where xxxxx is the catalog number for the assay in the MDS-Pharma Services on-line catalog found at URL “discovery.mdsps.com/catalog”. Thus, for example, “IC50-21950|Dopamine D1” indicates the Dopamine D1 assay with the MDS catalog number 21950. All compound activities are reported as −log(IC50), where the IC50 is reported in μM. Therefore, “>=0.000000000001” indicates that the value should be greater than zero and thus greater than 1 μm (i.e. since log(1 μM)=0). Furthermore, the testing protocols used in constructing the database of Example 1 did not determine IC50 values greater than about 35 μM. All cases where the IC50 was estimated to be greater than 35 μm was recorded in the database as “−3” (i.e. the IC50 was considered to be 1 nM and thus, −log(1000 μM)=−3). This number implies that the compound does not bind to the site under investigation.

b. Algorithms for Generating Valid Classifiers

Dataset classification may be carried out manually, that is by evaluating the dataset by eye and classifying the data accordingly. However, because the dataset may involve tens of thousands (or more) individual variables, more typically, querying the full dataset with a classification question is carried out in a computer employing any of the well-known data classification algorithms.

In preferred embodiments, algorithms are used to query the full dataset that generate linear classifiers. In particularly preferred embodiments the algorithm is selected from the group consisting of: SPLP, SPLR and SPMPM. These algorithms are based respectively on Support Vector Machines (SVM), Logistic Regression (LR) and Minimax Probability Machine (MPM). They have been described in detail elsewhere (See e.g., El Ghaoui et al., op. cit; Brown, M. P., W. N. Grundy, D. Lin, N. Cristianini, C. W. Sugnet, T. S. Furey, M. Ares, Jr., and D. Haussler, “Knowledge-based analysis of microarray gene expression data by using support vector machines,” Proc Natl Acad Sci U.S.A. 97: 262-267 (2000)).

Generally, the sparse classification methods SPLP, SPLR, SPMPM are linear classification algorithms in that they determine the optimal hyperplane separating a positive and a negative class. This hyperplane, H can be characterized by a vectorial parameter, w (the weight vector) and a scalar parameter, b (the bias): H={x|w^{T}x+=0}.

For all proposed algorithms, determining the optimal hyperplane reduces to optimizing the error on the provided training data points, computed according to some loss function (e.g., the “Hinge loss,” i.e., the loss function used in 1-norm SVMs; the “LR loss;” or the “MPM loss” augmented with a 1-norm regularization on the signature, w. Regularization helps to provide a sparse, short signature. Moreover, this 1-norm penalty on the signature will be weighted by the average standard error per gene. That is, genes that have been measured with more uncertainty will be less likely to get a high weight in the signature. Consequently, the proposed algorithms lead to sparse signatures, and take into account the average standard error information.

Mathematically, the algorithms can be described by the cost finctions (shown below for SPLP, SPLR and SPMPM) that they actually minimize to determine the parameters w and b.

$$\begin{matrix}{min}_{w , b} {\sum }_{i} {e}_{i} + ρ {\sum }_{i} {σ}_{i} \vert {w}_{i} \vert s . t . {y}_{i} ( {w}^{T} {x}_{i} + b ) \geq 1 - {e}_{i} \\ {e}_{i} \geq 0 , i = 1 , … , N\end{matrix} \qquad SPLP$$

The first term minimizes the training set error, while the second term is the 1-norm penalty on the signature w, weighted by the average standard error information per gene given by sigma. The training set error is computed according to the so-called Hinge loss, as defined in the constraints. This loss function penalizes every data point that is closer than “1” to the separating hyperplane H, or is on the wrong side of H. Notice how the hyperparameter rho allows trade-off between training set error and sparsity of the signature w.

$${min}_{w , b} {\sum }_{i} log ( 1 + exp ( - {y}_{i} ( {w}^{T} {x}_{i} + b ) ) ) + ρ {\sum }_{i} {σ}_{i} \vert {w}_{i} \vert \qquad SPLR$$

The first term expresses the negative log likelihood of the data (a smaller value indicating a better fit of the data), as usual in logistic regression, and the second term will give rise to a short signature, with rho determining the trade-off between both.

$${min}_{w} \sqrt{{w}^{T} {\hat{Γ}}_{+} w} + \sqrt{{w}^{T} {\hat{Γ}}_{-} w} + ρ {\sum }_{i} {σ}_{i} \vert {w}_{i} \vert s . t . {w}^{T} ( {\hat{x}}_{+} - {\hat{x}}_{-} ) = 1 \qquad SPMPM$$

Here, the first two terms, together with the constraint are related to the misclassification error, while the third term will induce sparsity, as before. The symbols with a hat are empirical estimates of the covariances and means of the positive and the negative class. Given those estimates, the misclassification error is controlled by determining w and b such that even for the worst-case distributions for the positive and negative class (which we do not exactly know here) with those means and covariances, the classifier will still perform well. More details on how this exactly relates to the previous cost function can be found in e.g., El Ghaoui et al., op. cit.

As mentioned above, classification algorithms capable of producing linear classifiers are preferred for use with the present invention. In the context of chemogenomic datasets, linear classifiers may be used to generate one or more valid signatures capable of answering a classification question comprising a series of genes and associated weighting factors. Linear classification algorithms are particularly useful with DNA array or proteomic datasets because they provide simplified signatures useful for answering a wide variety of questions related to biological function and pharmacological/toxicological effects associated with genes or proteins. These signatures are particularly useful because they are easily incorporated into wide variety of DNA- or protein-based diagnostic assays (e.g., DNA microarrays).

However, some classes of non-linear classifiers, so called kernel methods, may also be used to develop short gene lists, weights and algorithms that may be used in diagnostic device development; while the preferred embodiment described here uses linear classification methods, it specifically contemplates that non-linear methods may also be suitable.

Classifications may also be carried using principle component analysis and/or discrimination metric algorithms well-known in the art (see e.g., US 2003/0180808 A1, published Sep. 25, 2003, which is hereby incorporated by reference herein).

c. Cross-Validation of Classifiers

Cross-validation of signature performance is an important step for identifying sufficient signatures. Cross-validation may be carried out by first randomly splitting the full dataset (e.g., a 60/40 split). A training signature is derived from the training set composed of 60% of the samples and used to classify both the training set and the remaining 40% of the data, referred to herein as the test set. In addition, a complete signature is derived using all the data. The performance of these signatures can be measured in terms of log odds ratio (LOR) or the error rate (ER) defined as:
LOR=ln(((TP+0.5)*(TN+0.5))/((FP+0.5)*(FN+0.5)))
and
ER=(FP+FN)/N;

where TP, TN, FP, FN, and N are true positives, true negatives, false positives, false negatives, and total number of samples to classify, respectively, summed across all the cross validation trials. The performance measures are used to characterize the complete signature, the average of the training or the average of the test signatures.

The algorithms described above generate a plurality of classifiers with varying degrees of performance for the classification task. In order to identify valid classifiers, a threshold performance is set for an answer to the particular classification question. In one preferred embodiment, the classifier threshold performance is set as log odds ratio greater than or equal to 4.00 (i.e., LOR≧4.00). However, higher or lower thresholds may be used depending on the particular dataset and the desired properties of the classifiers so obtained. Of course many queries of the dataset with a classification will not generate a valid classifier.

Two or more valid signatures may be generated that are redundant or synonymous for a variety of reasons. Different classification questions (i.e., class definitions) may result in identical classes and therefore identical signatures. For instance, the following two class definitions define the exact same treatments in the database: (1) all treatments with molecules structurally related to statins; and (2) all treatments with molecules having an IC₅₀<1 μM for inhibition of the enzyme HMG CoA reductase.

In addition, when a large dataset is queried with the same classification question using different algorithms (or even the same algorithm under slightly different conditions) different, valid signatures may be obtained. These different signatures may or may not comprise overlapping sets of variables; however, they each can accurately identify members of the class of interest.

For example, as illustrated in Table 1, two equally performing gene signatures (LOR=˜7.0) for the fibrate class of compounds may be generated by querying a chemogenomic dataset with two different algorithms: SPLP and SPLR. Genes are designated by their accession number and a brief description. The weights associated with each gene are also indicated. Each signature was trained on the exact same 60% of the multivariate dataset and then cross validated on the exact same remaining 40% of the dataset. Both signatures were shown to exhibit the exact same level of performance as classifiers: two errors on the cross validation data set. The SPLP derived signature consists of 20 genes. The SPLR derived signature consists of eight genes. Only three of the genes from the SPLP signature are present in the eight gene SPLR signature.

TABLE 1
Two Gene Signatures for the Fibrate Class of Drugs

| | Accession | Weight | Unigene name |
|---|---|---|---|
| RLPC | K03249 | 1.1572 | enoyl-Co A, hydratase/3-hydroxyacyl |
| | | | Co A dehydrogenase |
| | AW916833 | 1.0876 | hypothetical protein RMT-7 |
| | BF387347 | 0.4769 | ESTs |
| | BF282712 | 0.4634 | ESTs |
| | AF034577 | 0.3684 | pyruvate dehydrogenate kinase 4 |
| | NM_019292 | 0.3107 | carbonic anhydrase 3 |
| | AI179988 | 0.2735 | ectodermal-neural cortex (with |
| | | | BTB-like domain) |
| | AI715955 | 0.211 | Stac protein (SRC homology 3 and |
| | | | cysteine-rich domain protein) |
| | BE110695 | 0.2026 | activating transcription factor 1 |
| | J03752 | 0.0953 | microsomal glutathione S-transferase 1 |
| | D86580 | 0.0731 | nuclear receptor subfamily 0, group B, |
| | | | member 2 |
| | BF550426 | 0.0391 | KDEL (Lys-Asp-Glu-Leu) endoplasmic |
| | | | reticulum protein retention receptor 2 |
| | AA818999 | 0.0296 | muscleblind-like 2 |
| | NM_019125 | 0.0167 | probasin |
| | AF150082 | −0.0141 | translocase of inner mitochondrial |
| | | | membrane 8 (yeast) homolog A |
| | BE118425 | −0.0781 | Arsenical pump-driving ATPase |
| | NM_017136 | −0.126 | squalene epoxidase |
| | AI171367 | −0.3222 | HSPC154 protein |
| | NM_019369 | −0.637 | inter alpha-trypsin inhibitor, heavy |
| | | | chain 4 |
| | AI137259 | −0.7962 | ESTs |
| SPLR | NM_017340 | 5.3688 | acyl-coA oxidase |
| | BF282712 | 4.1052 | ESTs |
| | NM_012489 | 3.8462 | acetyl-Co A acyltransferase 1 |
| | | | (peroxisomal 3-oxoacyl-Co A thiolase) |
| | BF387347 | 1.767 | ESTs |
| | K03249 | 1.7524 | enoyl-Co A, hydratase/3-hydroxyacyl |
| | | | Co A dehydrogenase |
| | NM_016986 | 0.0622 | acetyl-co A dehydrogenase, |
| | | | medium chain |
| | AB026291 | −0.7456 | acetoacetyl-CoA synthetase |
| | AI454943 | −1.6738 | likely ortholog of mouse porcupine |
| | | | homolog |

It is interesting to note that only three genes are common between these two signatures, (K03249, BF282712, and BF387347) and even those are associated with different weights. While many of the genes may be different, some commonalities may nevertheless be discerned. For example, one of the negatively weighted genes in the SPLP derived signature is NM_{—}017136 encoding squalene epoxidase, a well-known cholesterol biosynthesis gene. Squalene epoxidase is not present in the SPLR derived signature but aceto-acteylCoA synthetase, another cholesterol biosynthesis gene is present and is also negatively weighted.

Additional variant signatures may be produced for the same classification task. For example, the average signature length (number of genes) produced by SPLP and SPLR, as well as the other algorithms, may be varied by use of the parameter p (see e.g., El Ghaoui, L., G. R. G. Lanckriet, and G. Natsoulis, 2003, “Robust classifiers with interval data” Report# UCB/CSD-03-1279. Computer Science Division (EECS), University of California, Berkeley, Calif.; and U.S. provisional applications U.S. Ser. No. 60/495,975, filed Aug. 13, 2003 and U.S. Ser. No. 60/495,081, filed Aug. 13, 2003, each of which is hereby incorporated by reference herein). Varying ρ can produce signatures of different length with comparable test performance (Natsoulis et al., 2004, Gen. Res.). Those signatures are obviously different and often have no common genes between them (i.e., they do not overlap in terms of genes used).

C. Stripping Valid Classifiers to Generate the “Necessary” Variables

Each individual classifier or signature is capable of classifying a dataset into one of two categories or classes defined by the classification question. Typically, an individual signature with the highest test log odds ratio will be considered as the best classifier for a given task. However, often the second, third (or lower) ranking signatures, in terms of performance, may be useful for confirming the classification of compound treatment, especially where the unknown compound yields a borderline answer based on the best classifier. Furthermore, the additional signatures may identify alternative sources of informational rich data associated with the specific classification question. For example, a slightly lower ranking gene signature from a chemogenomic dataset may include those genes associated with a secondary metabolic pathway affected by the compound treatment. Consequently, for purposes of fully characterizing a class and answering difficult classification questions, it is useful to define the entire set of variables that may be used to produce the plurality of different classifiers capable of answering a given classification question. This set of variables is referred to herein as a “necessary set.” Conversely, the remaining variables from the full dataset are those that collectively cannot be used to produce a valid classifier, and therefore are referred to herein as the “depleted set.”

The general method for identifying a necessary set of variables useful for a classification question involved what is referred to herein as a classifier “stripping” algorithm. The stripping algorithm comprises the following steps: (1) querying the full dataset with a classification question so as to generate a first linear classifier capable of performing with a log odds ratio greater than or equal to 4.0 comprising a first set of variables; (2) removing the variables of the first linear classifier from the full dataset thereby generating a partially depleted dataset; (3) re-querying the partially depleted dataset with the same classification question so as to generate a second linear classifier and cross-validating this second classifier to determine whether it performs with a log odds ratio greater than or equal to 4. If it does not, the process stops and the dataset is fully depleted for variables capable of generating a classifier with an average log odds ratio greater than or equal to 4.0. If the second classifier is validated as performing with a log odds ratio greater than or equal to 4.0, then its variables are stripped from the full dataset and the partially depleted set if re-queried with the classification question. These cycles of stripping and re-querying are repeated until the performance of any remaining set of variables drops below an arbitrarily set LOR. The threshold at which the iterative process is stopped may be arbitrarily adjusted by the user depending on the desired outcome. For example, a user may choose a threshold of LOR=0. This is the value expected by chance alone. Consequently, after repeated stripping until LOR=0 there is no classification information remaining in the depleted set. Of course, selecting a lower value for the threshold will result in a larger necessary set.

Although a preferred cut-off for stripping classifiers is LOR=4.0, this threshold is arbitrary. Other embodiments within the scope of the invention may utilize higher or lower stripping cutoffs e.g., depending on the size or type of dataset, or the classification question being asked. In addition other metrics could be used to assess the performance (e.g., specificity, sensitivity, and others). Also the stripping algorithm removes all variables from a signature if it meets the cutoff. Other procedures may be used within the scope of the invention wherein only the highest weighted or ranking variables are stripped. Such an approach based on variable impact would likely result in a classifier “surviving” more cycles and defining a smaller necessary set.

The resulting fully-depleted set of variables that remains after a classifier is fully stripped from the full dataset cannot generate a classifier for the specific classification question (with the desired level of performance). Consequently, the set of all of the variables in the classifiers that were stripped from the full set are defined as “necessary” for generating a valid classifier.

The stripping method utilizes a classification algorithm at its core. The examples presented here use SPLP for this task. Other algorithms, provided that they are sparse with respect to genes could be employed. SPLR and SPMPM are two alternatives for this functionality (see e.g., El Ghaoui, L., G. R. G. Lanckriet, and G. Natsoulis, 2003, “Robust classifiers with interval data” Report# UCB/CSD-03-1279. Computer Science Division (EECS), University of California, Berkeley, Calif.; and U.S. provisional applications U.S. Ser. No. 60/495,975, filed Aug. 13, 2003 and U.S. Ser. No. 60/495,081, filed Aug. 13, 2003, each of which is hereby incorporated by reference herein).

In one embodiment, the stripping algorithm may be used on a chemogenomics dataset comprising DNA microarray data. The resulting necessary set of genes comprises a subset of highly informative genes for a particular classification question. Consequently, these genes may be incorporated in diagnostic devices (e.g., polynucleotide arrays) where that particular classification is of interest. In other exemplary embodiments, the stripping method may be used with datasets from a proteomic experiments.

Besides identifying the “necessary” set of variables for a classifier, another important use of the stripping algorithm is the identification of multiple, non-overlapping sufficient sets of variables useful as classifiers for a particular question. These non-overlapping sufficient sets are a direct product of the above-described general method of stripping valid classifiers. Where the application of the method results in a second validated classifier with the desired level of performance, that second classifier by definition does not include any variables in common with the first classifier. Typically, the earlier stripped non-overlapping classifiers yield higher performance with fewer variables. In other words, the earliest identified sufficient set usually comprises the highest impact, most information-rich variables with respect to the particular classification question. The valid classifiers that appear during the application of the stripping algorithm typically contain a larger number of variables. However, these later appearing classifiers may provide valuable information regarding normally unrecognized relationships between variables in the dataset. For example, in the case of non-overlapping gene signatures identified by stripping in a chemogenomics dataset, the later appearing signatures may include families of genes not previously recognized as involved in the particular metabolic pathway that is being affected by a particular compound treatment. Thus, functional analysis of a gene signature stripping procedure may identify new metabolic targets associated with a compound treatment.

D. Functional Characterization of Necessary Sets

The stripping method described herein produces a set of variables (e.g., genes) representing the information rich necessary set for a given classification question. Such necessary set, however, may be characterized in finctional terms based on the ability of the information rich genes in the set to supplement (i.e., “revive”) the ability of a fully depleted set to generate valid signatures for the classification question.

Thus, the necessary set for any classification question corresponds to that set of genes from which any random selection when added to a depleted set (i.e., depleted for that classification question) restores the ability of that set to produce signatures with an avg. LOR above a threshold level.

Preferably, the threshold performance is an avg. LOR greater than or equal to 4.00. Other values for performance, however, may be set. For example, avg. LOR may vary from about 1.0 to as high as 8.0. In preferred embodiments, the avg. LOR threshold may be 3.0 to as high as 7.0 including all integer and half-integer values in that range.

The necessary set may then be defined in terms of percentage of randomly selected genes from the necessary set that restore the performance of a depleted set above a certain threshold. Typically, the avg. LOR of the depleted set is ˜1.20, although as mentioned above, datasets may be depleted more or less depending on the threshold set, and depleted sets with avg. LOR as low as 0.0 may be used. Generally, the depleted set will exhibit an avg. LOR between about 0.5 and 1.5.

The third parameter establishing the functional characteristics of a specific necessary set of genes for answering a chemogenomic classification question is the percentage of randomly selected genes that results in restoring the threshold performance of the depleted set. Typically, where the threshold avg. LOR is at least 4.00 and the depleted set performs with an avg. LOR of ˜1.20, typically 16-36% of randomly selected genes from the necessary set are required to restore the average performance of the depleted set to the threshold value. In preferred embodiments, the random supplementation may be achieved using 16, 18, 20, 22, 24, 26, 28, 30, 32, 34 or 36% of the necessary set.

E. Diagnostic Assays and Reagent Sets Using Necessary and Sufficient Sets of Variables

As described above, a large dataset may be mined for a plurality of informative variables useful for answering classification questions. The size of the classifiers or signatures so generated may be varied according to experimental needs. In addition, multiple non-overlapping classifiers may be generated where independent experimental measures are required to confirm a classification. Generally, the necessary and sufficient sets of variables constitute a substantial reduction of data (i.e., relative to that present in the full data set), that needs to be measured to classify a sample. Consequently, the methods of the present invention provide the ability to produce cheaper, higher throughput, diagnostic measurement methods or strategies. In particular, the invention provides diagnostic reagent sets useful in diagnostic assays and the associated diagnostic devices and kits.

Diagnostic reagent sets may include reagents representing a select subset of sufficient variables consisting of less than 50%, 40%, 30%, 20%, 10%, or even less than 5% of the total analytical probes (i.e., detector moieties) present in a larger assay while still achieving the same level of performance in sample classification tasks. In one preferred embodiment, the diagnostic reagent set is a plurality of polynucleotides or polypeptides representing specific genes in a sufficient or necessary set of the invention. Such biopolymer reagent sets are immediately applicable in any of the diagnostic assay methods (and the associate kits) well known for polynucleotides and polypeptides (e.g., DNA arrays, RT-PCR, immunoassays or other receptor based assays for polypeptides or proteins). For example, by selecting only those genes found in a smaller yet “sufficient” gene signature, a faster, simpler and cheaper DNA array may be fabricated for that signature's specific classification task. Thus, a very simple diagnostic array may be designed that answers 3 or 4 specific classification questions and includes only 60-80 polynucleotides representing the approximately 20 genes in each of the signatures. Of course, depending on the level of accuracy required the LOR threshold for selecting a sufficient gene signature may be varied. A DNA array may be designed with many more genes per signature if the LOR threshold is set at e.g., 7.00 for a given classification question. The scope of the present invention includes diagnostic devices based on classifiers exhibiting levels of performance varying from less than LOR=3.00 up to LOR=10.00 and greater.

The diagnostic reagent sets of the invention may be provided in kits, wherein the kits may or may not comprise additional reagents or components necessary for the particular diagnostic application in which the reagent set is to be employed. Thus, for a polynucleotide array applications, the diagnostic reagent sets may be provided in a kit which further comprises one or more of the additional requisite reagents for amplifying and/or labeling a microarray probe or target (e.g., polymerases, labeled nucleotides, and the like).

A variety of array formats (for either polynucleotides and/or polypeptides) are well-known in the art and may be used with the methods and subsets produced by the present invention. In one preferred embodiment, photolithographic or micromirror methods may be used to spatially direct light-induced chemical modifications of spacer units or functional groups resulting in attachment at specific localized regions on the surface of the substrate. Light-directed methods of controlling reactivity and immobilizing chemical compounds on solid substrates are well-known in the art and described in U.S. Pat. Nos. 4,562,157, 5,143,854, 5,556,961, 5,968,740, and 6,153,744, and PCT publication WO 99/42813, each of which is hereby incorporated by reference herein.

Alternatively, a plurality of molecules may be attached to a single substrate by precise deposition of chemical reagents. For example, methods for achieving high spatial resolution in depositing small volumes of a liquid reagent on a solid substrate are disclosed in U.S. Pat. Nos. 5,474,796 and 5,807,522, both of which are hereby incorporated by reference herein.

It should also be noted that in many cases a single diagnostic device may not satisfy all needs. However, even for an initial exploratory investigation (e.g., classifying drug-treated rats) DNA arrays with sufficient gene sets of varying size (number of genes), each adapted to a specific follow-up technology, can be created. In addition, in the case of drug-treated rats, different arrays may be defined for each tissue.

Alternatively, a single substrate may be produced with several different small arrays of genes in different areas on the surface of the substrate. Each of these different arrays may represent a sufficient set of genes for the same classification question but with a different optimal gene signature for each different tissue. Thus, a single array could be used for particular diagnostic question regardless of the tissue source of the sample (or even if the sample was from a mixture of tissue sources, e.g., in a forensic sample).

In addition, it may be desirable to investigate classification questions of a different nature in the same tissue using several arrays featuring different non-overlapping gene signatures for a particular classification question.

As described above, the methodology described here is not limited to chemogenomic datasets and DNA microarray data. The invention may be applied to other types of datasets to produce necessary and sufficient sets of variables useful for generating classifiers. For example, proteomics assay techniques, where protein levels are measured or protein interaction techniques such as yeast 2-hybrid or mass spectrometry also result in large, highly multivariate dataset, which could be classified in the same way described here. The result of all the classification tasks could be submitted to the same methods of signature generation and/or classifier stripping in order to define specific sets of proteins useful as signatures for specific classification questions.

In addition, the invention is useful for many traditional lower throughput diagnostic applications. Indeed the invention teaches methods for generating valid, high-performance classifiers consisting of 5% or less of the total variables in a dataset. This data reduction is critical to providing a useful analytical device. For example, a large chemogenomic dataset may be reduced to a signature comprising less than 5% of the genes in the full dataset. Further reductions of these genes may be made by identifying only those genes whose product is a secreted protein. These secreted proteins may be identified based on known annotation information regarding the genes in the subset. Because the secreted proteins are identified in the sufficient set useful as a signature for a particular classification question, they are most useful in protein based diagnostic assays related to that classification. For example, an antibody-based blood serum assay may be produced using the subset of the secreted proteins found in the sufficient signature set. Hence, the present invention may be used to generate improved protein-based diagnostic assays from DNA array information.

The general method of the invention as described above is exemplified below. The following examples are offered by way of illustration and not by way of limitation. The disclosure of all citations in the specification is expressly incorporated herein by reference.

### EXAMPLE 1

This example illustrates the construction of a large multivariate chemogenomic dataset based on DNA microarray analysis of rat tissues from over 580 different in vivo compound treatments (311 of which were tested in liver). This dataset was used to generate signatures comprising genes and weights which subsequently were reduced to yield a subsets of highly responsive genes that may be incorporated into high throughput diagnostic devices as described in Examples 2-5.

The detailed description of the construction of this chemogenoric dataset is described in Examples 1 and 2 of Published U.S. patent application No. 2005/0060102 A1, published Mar. 17, 2005, which is hereby incorporated by reference for all purposes. Briefly, in vivo short-term repeat dose rat studies were conducted on over 580 test compounds, including marketed and withdrawn drugs, environmental and industrial toxicants, and standard biochemical reagents. Rats (three per group) were dosed daily at either a low or high dose. The low dose was an efficacious dose estimated from the literature and the high dose was an empirically-determined maximum tolerated dose, defined as the dose that causes a 50% decrease in body weight gain relative to controls during the course of the 5 day range finding study. Animals were necropsied on days 0.25, 1, 3, and 5 or 7. Up to 13 tissues (e.g., liver, kidney, heart, bone marrow, blood, spleen, brain, intestine, glandular and nonglandular stomach, lung, muscle, and gonads) were collected for histopathological evaluation and microarray expression profiling on the Amersham CodeLink™ RU1 platform. In addition, a clinical pathology panel consisting of 37 clinical chemistry and hematology parameters was generated from blood samples collected on days 3 and 5.

In order to assure that all of the dataset is of high quality a number of quality metrics and tests are employed. Failure on any test results in rejection of the array and exclusion from the data set. The first tests measure global array parameters: (1) average normalized signal to background, (2) median signal to threshold, (3) fraction of elements with below background signals, and (4) number of empty spots. The second battery of tests examines the array visually for unevenness and agreement of the signals to a tissue specific reference standard formed from a number of historical untreated animal control arrays (correlation coefficient >0.8). Arrays that pass all of these checks are further assessed using principle component analysis versus a dataset containing seven different tissue types; arrays not closely clustering with their appropriate tissue cloud are discarded.

Data collected from the scanner is processed by the Dewarping/Detrending™ normalization technique, which uses a non-linear centralization normalization procedure (see, Zien, A., T. Aigner, R. Zimmer, and T. Lengauer. 2001. Centralization: A new method for the normalization of gene expression data. Bioinformatics) adapted specifically for the CodeLink microarray platform. The procedure utilizes detrending and dewarping algorithms to adjust for non-biological trends and non-linear patterns in signal response, leading to significant improvements in array data quality.

Log₁₀-ratios are computed for each gene as the difference of the averaged logs of the experimental signals from (usually) three drug-treated animals and the averaged logs of the control signals from (usually) 20 mock vehicle-treated animals. To assign a significance level to each gene expression change, the standard error for the measured change between the experiments and controls is computed. An empirical Bayesian estimate of standard deviation for each measurement is used in calculating the standard error, which is a weighted average of the measurement standard deviation for each experimental condition and a global estimate of measurement standard deviation for each gene determined over thousands of arrays (Carlin, B. P. and T. A. Louis. 2000. “Bayes and empirical Bayes methods for data analysis, ” Chapman & Hall/CRC, Boca Raton; Gelman, A. 1995. “Bayesian data analysis, ” Chapman & Hall/CRC, Boca Raton). The standard error is used in a t-test to compute a p-value for the significance of each gene expression change. The coefficient of variation (CV) is defined as the ratio of the standard error to the average Log₁₀-ratio, as defined above.

### EXAMPLE 2

This example illustrates the use of the “stripping” method to define the necessary and depleted sets of genes for a chemogenomic classification question.

Stripping algorithm

For each of the 101 classification questions defined by Table 2, the full chemogenomic dataset made according to Example 1 was labeled (i.e., +1, −1, or 0). The labeled dataset was then queried using the SPLP algorithm until it produced a valid signature, defined as performing with a test LOR≧4.0. Then all of the genes of from the first valid signature were eliminated (i.e., “stripped”) from the full dataset. This now partially depleted dataset was then queried with the SPLP algorithm again until a second cross validated signature was computed applying the SPLP algorithm to the partially depleted dataset. If this second signature was valid, i.e., performed with a test LOR≧4.0, all of its genes were stripped from the full dataset. This process was repeated until the algorithm failed to produce a valid signature. The union set of all the “stripped” genes used in the valid signatures constituted the “necessary set.”

TABLE 2
101 Classification Questions

| | | | | | |
|---|---|---|---|---|---|
| | | | | Class-1 | Class 0 |
| No. | Classification Name | Universe Description | Class 1 description | description | description |

62 Classification Questions that Fail to Yield Valid Signatures After Four Stripping Cycles

| | | | | | |
|---|---|---|---|---|---|
| 1 | Monoamine Re-uptake | (Tissue = LIVER And | (STRUCTURE_ACTIVITY = Monoamine | All else | (Zero_Class = *** |
| | (SERT) inhibitor, | HighOrLowDose = HI) Not | Re-uptake (SERT) | | Blank***) |
| | heterogeneous structures IN | (STRUCTURE_ACTIVITY = *** | inhibitor, heterogeneous structures | | Or (Zero_Class = Y) |
| | LIVER | Blank***) | | | |
| 2 | Estrogen antagonist, | (Tissue = LIVER And | (STRUCTURE_ACTIVITY = Estrogen | All else | (Zero_Class = *** |
| | aromatase inhibitor IN | HighOrLowDose = HI) Not | antagonist, aromatase | | Blank***) |
| | LIVER | (STRUCTURE_ACTIVITY = *** | inhibitor | | Or (Zero_Class = Y) |
| | | Blank***) | | | |
| 3 | PXR_liver_NoDEX+1_specific- | (Tissue = LIVER) | (PXR_Class_1_NO_DEX = YES) | (PXR_negative_specific = YES) | All else |
| | 1_MIFE | | | Or | |
| | | | | (mifepristone | |
| | | | | included = EITHER + OR) | |
| 4 | DNA-alkylator IN LIVER | (Tissue = LIVER And | (STRUCTURE_ACTIVITY = DNA- | All else | (Zero_Class = *** |
| | | TimePoint >= 3) Not | alkylator | | Blank***) |
| | | (STRUCTURE_ACTIVITY = *** | | | Or (Zero_Class = Y) |
| | | Blank***) | | | |
| 5 | Embryotoxicity IN LIVER | (Tissue = LIVER) Not | (TISSUE_TOXICITY = Embryotoxicity) | All else | (Zero_Class = *** |
| | | (TISSUE_TOXICITY = *** | | | Blank***) |
| | | Blank***) | | | Or (Zero_Class = Y) |
| 6 | GABAA, Benzodiazepine, | (Tissue = LIVER) Not (IC50- | (IC50-22660\|GABAA, | All else | (MDS_Specific_Groupings_{—} |
| | timed 10 uM | 22660\|GABAA, | Benzodiazepine, Central >= −1 And | | A = GABA_agonist_{—} |
| | | Benzodiazepine, Central = *** | MDS_Specific_Groupings_A = GABA_{—} | | channel) Or |
| | | Blank***) | agonist_timed) | | (New_Activity_Class = |
| | | | | | GABA-B agonist) |
| 7 | IC50-22032\|Dopamine | (Tissue = LIVER) | >=0.0000000000001 | −3 | All else |
| | Transporter | | | | |
| 8 | Later timepoints CAR | (Tissue = LIVER And | see KK109, long term | ALL ELSE | BLIND, |
| | ligands | TimePoint >= 3 but <= 5) | benzodiazepines nad phenobarbital | | AVENTIS |
| | | | and estrogens | | |
| 9 | Pro-inflammatory stimuli IN | (Tissue = LIVER) Not | (STRUCTURE_ACTIVITY = Pro- | All else | (Zero_Class = *** |
| | LIVER | (STRUCTURE_ACTIVITY = *** | inflammatory stimuli | | Blank***) |
| | | Blank***) | | | Or (Zero_Class = Y) |
| 10 | Testosterone_agonist c | (Tissue = LIVER) Not (IC50- | (IC50-28501\|Testosterone >= 0 And | (IC50- | All else |
| | | 28501\|Testosterone = *** | MDS_Specific_Groupings_A = Androgen_{—} | 28501\|Testosterone = −3) | |
| | | Blank***) | agonist) Not | Or | |
| | | | (MDS_Specific_Groupings_A = Androgen_{—} | (MDS_Specific_{—} | |
| | | | antagonist) | Groupings_A = Androgen_{—} | |
| | | | | antagonist) | |
| 11 | phospholipidosis_liver_not_{—} | (Tissue = LIVER) | (PHOSPHOLIPIDOSIS = Y) Not | All else | (Drug = FLUOXETINE) |
| | fluoxetine | | (Drug = FLUOXETINE) | | |
| 12 | Progesterone receptor | (Tissue = LIVER And | (ACTIVITY_CLASS_UNION = Progesterone | All else | (Zero_Class = *** |
| | agonist IN LIVER | HighOrLowDose = HI) Not | receptor agonist | | Blank***) |
| | | (ACTIVITY_CLASS_UNION = *** | | | Or (Zero_Class = Y) |
| | | Blank***) | | | |
| 13 | IC50-21460\|Calcium | (Tissue = LIVER) | >=0.0000000000001 | −3 | All else |
| | Channel Type L, | | | | |
| | Dihydropyridine | | | | |
| 14 | IC50-17110\|Protein | (Tissue = LIVER) | >=0.0000000000001 | −3 | All else |
| | Serine/Threonine Kinase, | | | | |
| | ERK2 | | | | |
| 15 | HistoCont_LIVER_(3, 5, | (TISSUE = LIVER And | LIVER-HEPATOCYTE | LIVER- | all else |
| | 7)_LIVER-HEPATOCYTE | TimePoint >= 3 but <= 7 And | ENLARGEMENT SEVERITY | HEPATOCYTE | |
| | ENLARGEMENT_(>2_3_animal) | LIVER-HEPATOCYTE | SCORE > 2 in at least 3 animal(s) | ENLARGEMENT | |
| | | ENLARGEMENT = Y) | | SEVERITY | |
| | | | | SCORE = 0 in all | |
| | | | | animals | |
| 16 | Toxicant, free oxygen | (Tissue = LIVER) Not | (STRUCTURE_ACTIVITY = Toxicant, | All else | (Zero_Class = *** |
| | radical generator IN LIVER | (STRUCTURE_ACTIVITY = *** | free oxygen radical | | Blank***) |
| | | Blank***) | generator | | Or (Zero_Class = Y) |
| 17 | DNA damaging, free oxygen | (Tissue = LIVER And | (STRUCTURE_ACTIVITY = DNA | All else | (Zero_Class = *** |
| | radical generator, | TimePoint >= 3) Not | damaging, free oxygen radical | | Blank***) |
| | nitrosourea IN LIVER | (STRUCTURE_ACTIVITY = *** | generator, nitrosourea | | Or (Zero_Class = Y) |
| | | Blank***) | | | |
| 18 | ALB_UP_SIG_LI_2% | (Tissue = LIVER And | 98th percentile; liver; day5/7 | 0-75th percentile; | other |
| | | TimePoint >= 5 And | | liver; day5/7 | |
| | | ClinicalChemInfo = Y) | | | |
| 19 | ClinSpecContDecr_LIVER_{—} | (TISSUE = LIVER And | Day5_Logratio_TBI + Logratio_{—} | Logratio TBI + Logratio_{—} | all else |
| | (3)_Logratio_TBI + Logratio_{—} | TimePoint = 3 And | ALP + Logratio_ALT <= 5th | ALP + Logratio_{—} | |
| | ALP + Logratio_{—} | Day5_Logratio_TBI + Logratio_{—} | percentile | ALT >= 35th | |
| | ALT_(5, 35, 0) | ALP + Logratio_ALT = Y) | | percentile | |
| 20 | Dopamine D1_antagonist a | (Tissue = LIVER) Not (IC50- | (IC50-21950\|Dopamine D1 >= 0) | (IC50- | All else |
| | | 21950\|Dopamine D1 = *** | Not (MDS_Specific_Groupings_A = D_{—} | 21950\|Dopamine | |
| | | Blank***) | agonist) | D1 = −3) Or | |
| | | | | (MDS_Specific_{—} | |
| | | | | Groupings_A = D_{—} | |
| | | | | agonist) | |
| 21 | IC50-21500\|Calcium | (Tissue = LIVER) | >=0.0000000000001 | −3 | All else |
| | Channel Type L, | | | | |
| | Phenylalkylamine | | | | |
| 22 | DNA damaging, free oxygen | (Tissue = LIVER) Not | (STRUCTURE_ACTIVITY = DNA | All else | (Zero_Class = *** |
| | radical generator IN LIVER | (STRUCTURE_ACTIVITY = *** | damaging, free oxygen radical | | Blank***) |
| | | Blank***) | generator | | Or (Zero_Class = Y) |
| 23 | Estrogen | (Tissue = LIVER) Not (IC50- | (IC50-22601\|Estrogen ERalpha >= 0) | (IC50- | All else |
| | ERalpha_antagonist a | 22601\|Estrogen ERalpha = *** | Not | 22601\|Estrogen | |
| | | Blank***) | (MDS_Specific_Groupings_A = Estrogen_{—} | ERalpha = −3) Or | |
| | | | agonist) | (MDS_Specific_{—} | |
| | | | | Groupings_A = Estrogen_agonist) | |
| 24 | Bacterial ribosomal (50S) | (Tissue = LIVER And | (STRUCTURE_ACTIVITY = Bacterial | All else | (Zero_Class = *** |
| | function inhibitor, macrolide | HighOrLowDose = HI) Not | ribosomal (50S) function | | Blank***) |
| | IN LIVER | (STRUCTURE_ACTIVITY = *** | inhibitor, macrolide | | Or (Zero_Class = Y) |
| | | Blank***) | | | |
| 25 | Dopamine receptor | (Tissue = LIVER) Not | (STRUCTURE_ACTIVITY = Dopamine | All else | (Zero_Class = *** |
| | antagonist (D), | (STRUCTURE_ACTIVITY = *** | receptor antagonist (D), | | Blank***) |
| | phenothiazine IN LIVER | Blank***) | phenothiazine | | Or (Zero_Class = Y) |
| 26 | Estrogen antagonist, | (Tissue = LIVER) Not | (ACTIVITY_CLASS_UNION = Estrogen | All else | (Zero_Class = *** |
| | aromatase | (ACTIVITY_CLASS_UNION = *** | antagonist, aromatase | | Blank***) |
| | inhibitor_Estrogen receptor | Blank***) | inhibitor_Estrogen receptor | | Or (Zero_Class = Y) |
| | antagonist/agonist, tissue | | antagonist/agonist, tissue specific | | |
| | specific IN LIVER | | | | |
| 27 | Ca++ channel (L-Type) | (Tissue = LIVER) Not | (ACTIVITY_CLASS_UNION = Ca++ | All else | (Zero_Class = *** |
| | blocker_Ca++ channel (L- | (ACTIVITY_CLASS_UNION = *** | Ca++ channel (L-Type) | | Blank***) |
| | Type) blocker, 1,4- | Blank***) | blocker_Ca++ channel (L-Type) | | Or (Zero_Class = Y) |
| | DHP_Ca++ channel (T- | | blocker, 1,4-DHP_Ca++ channel (T- | | |
| | Type) blocker_Ca++ | | Type) blocker_Ca++ channel | | |
| | channel blocker, | | blocker, antiparasitics | | |
| | antiparasitics IN LIVER | | | | |
| 28 | HistoCont_LIVER_(5, | (TISSUE = LIVER And | LIVER-FATTY CHANGE | LIVER-FATTY | all else |
| | 7)_LIVER-FATTY | TimePoint >= 5 but <= 7 And | SEVERITY SCORE > 2 in at least 3 | CHANGE | |
| | CHANGE_(>2_3_animal) | LIVER-FATTY CHANGE = Y) | animal(s) | SEVERITY | |
| | | | | SCORE = 0 in all | |
| | | | | animals | |
| 29 | Sterol 14-demethylase | (Tissue = LIVER And | (ACTIVITY_CLASS_UNION = Sterol | All else | (Zero_Class = *** |
| | inhibitor_Sterol 14- | HighOrLowDose = HI) Not | 14-demethylase | | Blank***) |
| | demethylase inhibitor, | (ACTIVITY_CLASS_UNION_= *** | inhibitor_Sterol 14-demethylase | | Or (Zero_Class = Y) |
| | ketoconazole like_Sterol 14- | Blank***) | inhibitor, ketoconazole like_Sterol | | |
| | demethylase inhibitor, | | 14-demethylase inhibitor, | | |
| | miconazole like IN LIVER | | miconazole like | | |
| 30 | AP_UP_SIG_LI_2%_B | (Tissue = LIVER And | 98th percentile; liver; day5/7 | 25-75th | other |
| | | TimePoint >= 5 And | | percentile; liver; | |
| | | ClinicalChemInfo = Y) | | day5/7 | |
| 31 | ClinPredDecr_LIVER_(0.25 | (TISSUE = LIVER And | Day5_LIPASE <= 5th percentile | Day5_LIPASE <= 65th | all else |
| | )_LIPASE_(5, 35, 65) | TimePoint = 0.25 And | | percentile And | |
| | | Day5_LIPASE = Y) | | Day5_LIPASE >= 35th | |
| | | | | percentile | |
| 32 | LI_HEMOGLOBIN_DECREASE_>=5 hr | (Tissue = LIVER And | 98th % | 25-75th % | rest |
| | | TimePoint >= 5 And | | | |
| | | ClinicalChemInfo = Y) | | | |
| 33 | HistoPredSum_LIVER_(0.25, | (TISSUE = LIVER And | Day5_LIVER-NECROSIS SUM OF | Day5_LIVER- | all else |
| | 1)_LIVER- | TimePoint >= 0.25 but <= 1 And | SEVERITY SCORE > 2 | NECROSIS | |
| | NECROSIS_SUM_OF_SEVERITY > 2 | Day5_LIVER-NECROSIS = Y) | | SUM OF | |
| | | | | SEVERITY | |
| | | | | SCORE = 0 | |
| 34 | 5HT2/D4/D2 antagonist, | (Tissue = LIVER And | (ACTIVITY_CLASS_UNION = 5HT2/ | All else | (Zero_Class = *** |
| | tricyclic | TimePoint >= 3) Not | D4/D2 antagonist, tricyclic | | Blank***) |
| | antipsychotic_5HT2/D4/D2 | (ACTIVITY_CLASS_UNION = *** | antipsychotic_5HT2/D4/D2 | | Or (Zero_Class = Y) |
| | antagonist, tricyclic | Blank***) | antagonist, tricyclic | | |
| | antipsychotic_5HT2/H1 | | antipsychotic_5HT2/H1 antagonist, | | |
| | antagonist, tricyclic_5HT3 | | tricyclic_5HT3 antagonist | | |
| | antagonist IN LIVER | | | | |
| 35 | IC50-21755\|Chemokine | (Tissue = LIVER) | >−1 Not ***Blank*** | −3 | All else |
| | CCR2B | | | | |
| 36 | LI_HEMATOCRIT_INCREASE_>=5 hr | (Tissue = LIVER And | 98th % | 25-75th % | rest |
| | | TimePoint >= 5 And | | | |
| | | ClinicalChemInfo = Y) | | | |
| 37 | NSAID, COX-2/1, coxib | (Tissue = LIVER And | (STRUCTURE_ACTIVITY = NSAID, | All else | (Zero_Class = *** |
| | like IN LIVER | HighOrLowDose = HI) Not | COX-2/1, coxib like | | Blank***) |
| | | (STRUCTURE_ACTIVITY = *** | | | Or (Zero_Class = Y) |
| | | Blank***) | | | |
| 38 | Hepatocellular Carcinoma | (Tissue = LIVER) Not | (TISSUE_TOXICITY = Hepatocellular | All else | (Zero_Class = *** |
| | IN LIVER | (TISSUE_TOXICITY = *** | Carcinoma) | | Blank***) |
| | | Blank***) | | | Or (Zero_Class = Y) |
| 39 | NSAID, COX-1_NSAID, | (Tissue = LIVER And | (ACTIVITY_CLASS_UNION = NSAID, | All else | (Zero_Class = *** |
| | COX-1, 6-Methoxy- | HighOrLowDose = HI) Not | COX-1_NSAID, COX-1, 6- | | Blank***) |
| | naphthalenyl-acetic | (ACTIVITY_CLASS_UNION = *** | Methoxy-naphthalenyl-acetic | | Or (Zero_Class = Y) |
| | acid_NSAID, COX-1, | Blank***) | acid_NSAID, COX-1, | | |
| | arylacylprofen_NSAID, | | arylacylprofen_NSAID, COX-1, | | |
| | COX-1, ibuprofen | | ibuprofen like_NSAID, COX-1, | | |
| | like_NSAID, COX-1, | | indomethacin like | | |
| | indomethacin like IN | | | | |
| | LIVER | | | | |
| 40 | IC50-28501\|Testosterone | (Tissue = LIVER) | >=0.0000000000001 | −3 | All else |
| 41 | GABA-A agonist, | (Tissue = LIVER And | (STRUCTURE_ACTIVITY = GABA-A | All else | (Zero_Class = *** |
| | benzodiazepin, long acting | HighOrLowDose = HI And | agonist, benzodiazepin, | | Blank***) |
| | IN LIVER | TimePoint >= 3) Not | long acting | | Or (Zero_Class = Y) |
| | | (STRUCTURE_ACTIVITY = *** | | | |
| | | Blank***) | | | |
| 42 | IC50-26011\|Opiate delta | (Tissue = LIVER) | >−1 Not ***Blank*** | −3 | All else |
| 43 | REL_LIVER_WT_UP_SIG_{—} | (Tissue = LIVER And | 98th percentile; liver; day5/7 | 0-75th percentile; | other |
| | LI_2% | TimePoint >= 5 And | | liver; day5/7 | |
| | | Organ_Weight_Info = Y) | | | |
| 44 | HistoPred_LIVER_(0.25, | (TISSUE = LIVER And | Day5_LIVER-NECROSIS | Day5_LIVER- | all else |
| | 1)_LIVER- | TimePoint >= 0.25 but <= 1 And | SEVERITY SCORE > 0 in at least 2 | NECROSIS | |
| | NECROSIS_(>0_2_animal) | Day5_LIVER-NECROSIS = Y) | animal(s) | SEVERITY | |
| | | | | SCORE = 0 in all | |
| | | | | animals | |
| 45 | ClinContDecr_LIVER_(3, 5, | (TISSUE = LIVER And | LYMPHOCYTE <= 5th percentile | LYMPHOCYTE >= 35th | all else |
| | 7)_LYMPHOCYTE_(5, 35, 0) | TimePoint >= 3 but <= 7 And | | percentile | |
| | | LYMPHOCYTE = Y) | | | |
| 46 | IC50-27191\|Serotonin 5- | (Tissue = LIVER) | >−1 Not ***Blank*** | −3 | All else |
| | HT3 | | | | |
| 47 | IC50-20420\|Adrenergic | (Tissue = LIVER) | >−1 Not ***Blank*** | −3 | All else |
| | beta3 | | | | |
| 48 | Bacterial ribosomal (30S) | (Tissue = LIVER And | (STRUCTURE_ACTIVITY = Bacterial | All else | (Zero_Class = *** |
| | function inhibitor, | TimePoint >= 3) Not | ribosomal (30S) function | | Blank***) |
| | tetracycline IN LIVER | (STRUCTURE_ACTIVITY = *** | inhibitor, tetracycline | | Or (Zero_Class = Y) |
| | | Blank***) | | | |
| 49 | IC50-27820\|Sigma2 | (Tissue = LIVER) | >=0.0000000000001 | −3 | All else |
| 50 | ClinContDecr_LIVER_(3, 5, | (TISSUE = LIVER And | LEUKOCYTE COUNT <= 5th | LEUKOCYTE | all else |
| | 7)_LEUKOCYTE | TimePoint >= 3 but <= 7 And | percentile | COUNT >= 35th | |
| | COUNT_(5, 35, 0) | LEUKOCYTE COUNT = Y) | | percentile | |
| 51 | Estrogen receptor agonist, | (Tissue = LIVER) Not | (STRUCTURE_ACTIVITY = Estrogen | All else | (Zero_Class = *** |
| | environmental toxicant IN | (STRUCTURE_ACTIVITY = *** | receptor agonist, | | Blank***) |
| | LIVER | Blank***) | environmental toxicant | | Or (Zero_Class = Y) |
| 52 | IC50-27951\|Sodium | (Tissue = LIVER) | >=0.0000000000001 | −3 | All else |
| | Channel, Site 2 | | | | |
| 53 | Muscarinic M2_antagonistse | (Tissue = LIVER) Not (IC50- | (IC50-25270\|Muscarinic M2 >= 0) | (IC50- | All else |
| | | 25270\|Muscarinic M2 = *** | Not (New_Activity_Class_Unions = Muscarinic | 25270\|Muscarinic | |
| | | Blank***) | acetylcoline receptor | M2 = −3) Or | |
| | | | (M) agonist) | (New_Activity_Class_{—} | |
| | | | | Unions = Muscarinic | |
| | | | | acetylcoline | |
| | | | | receptor (M) | |
| | | | | agonist) | |
| 54 | PXR_liver_all HI+1_ligand | (Tissue = LIVER) | (PXR_Class_1_DOSE = HI) | (PXR_negative_ligand_{—} | All else |
| | −1 | | | CYP3A_inhibitors_{—} | |
| | | | | literature = YES) | |
| 55 | Bacterial folate synthesis | #VALUE! | #VALUE! | #VALUE! | #VALUE! |
| | inhibitor, dihydropteroate | | | | |
| | synthase inhibitor_Bacterial | | | | |
| | folate synthesis inhibitor, | | | | |
| | dihydropteroate synthase | | | | |
| | inhibitor, isoxazol- | | | | |
| | sulfonamide_Bacterial folate | | | | |
| | synthesis inhibitor, | | | | |
| | dihydropteroate synthase | | | | |
| | inhibitor, pyrimidin- | | | | |
| | sulfonamide IN LIVER | | | | |
| 56 | Estrogen ERalpha_agonist d | (Tissue = LIVER) Not (IC50- | (IC50-22601\|Estrogen ERalpha >= −1 | (IC50- | All else |
| | | 22601\|Estrogen ERalpha = *** | And MDS_Specific_Groupings_A = Estrogen_{—} | 22601\|Estrogen | |
| | | Blank***) | agonist) Not | ERalpha = −3) Or | |
| | | | (MDS_Specific_Groupings_A = Estrogen_{—} | (MDS_Specific_{—} | |
| | | | antagonist) | Groupings_A = Estrogen_{—} | |
| | | | | antagonist) | |
| 57 | IC50-20051\|Adenosine A1 | (Tissue = LIVER) | >−1 Not ***Blank*** | −3 | All else |
| 58 | ClinSpecContIncr_LIVER_{—} | (TISSUE = LIVER And | Day5_Logratio_ALP + Logratio_{—} | Logratio_ALP + Logratio_{—} | all else |
| | (0.25, 1, 3, 5, | TimePoint >= 0.25 but <= 7 And | ALT >= 90th percentile | ALT <= 60th | |
| | 7)_Logratio_ALP + Logratio_{—} | Day5_Logratio_ALP + Logratio_{—} | | percentile | |
| | ALT_(90, 0, 60) | ALT = Y) | | | |
| 59 | IC50-19401\|Thromboxane | (Tissue = LIVER) | >=0.0000000000001 | −3 | All else |
| | Synthase | | | | |
| 60 | LI_LEUKOCYTE_COUNT_{—} | (Tissue = LIVER And | 95th % | 0-75th % | rest |
| | INCREASE on Day5_0.25 | TimePoint <= 1 And | | | |
| | or 1 | ClinicalChemInfo = Y) | | | |
| 61 | ClinContIncr_LIVER_(5, | (TISSUE = LIVER And | ABSOLUTE SEGMENTED | ABSOLUTE | all else |
| | 7)_ABSOLUTE | TimePoint >= 5 but <= 7 And | NEUTROPHIL >= 95th percentile | SEGMENTED | |
| | SEGMENTED | ABSOLUTE SEGMENTED | | NEUTROPHIL <= 65th | |
| | NEUTROPHIL_(95, 35, 65) | NEUTROPHIL = Y) | | percentile And | |
| | | | | ABSOLUTE | |
| | | | | SEGMENTED | |
| | | | | NEUTROPHIL >= 35th | |
| | | | | percentile | |
| 62 | LI_CREATININE_INCREASE_5 | (Tissue = LIVER And | 95th % | 0-75th % | rest |
| | | TimePoint >= 5 And | | | |
| | | ClinicalChemInfo = Y) | | | |

39 Classification Questions that Continue to Produce Valid Signatures After 4 Stripping Cycles

| | | | | | |
|---|---|---|---|---|---|
| 1 | HMG-CoA reductase | (Tissue = LIVER And | (STRUCTURE_ACTIVITY = HMG- | All else | (Zero_Class = *** |
| | inhibitors IN LIVER | HighOrLowDose = HI And | CoA reductase inhibitors | | Blank***) |
| | | TimePoint >= 3) Not | | | Or (Zero_Class = Y) |
| | | (STRUCTURE_ACTIVITY = *** | | | |
| | | Blank***) | | | |
| 2 | Estrogen receptor | (Tissue = LIVER And | (ACTIVITY_CLASS_UNION = Estrogen | All else | (Zero_Class = *** |
| | agonist_Estrogen receptor | TimePoint >= 3) Not | receptor agonist_Estrogen | | Blank***) |
| | agonist, steroidal IN LIVER | (ACTIVITY_CLASS_UNION = *** | receptor agonist, steroidal | | Or (Zero_Class = Y) |
| | | Blank***) | | | |
| 3 | Estrogen receptor | (Tissue = LIVER And | (STRUCTURE_ACTIVITY = Estrogen | All else | (Zero_Class = *** |
| | antagonist/agonist, tissue | TimePoint >= 3) Not | receptor antagonist/agonist, | | Blank***) |
| | specific IN LIVER | (STRUCTURE_ACTIVITY = *** | tissue specific | | Or (Zero_Class = Y) |
| | | Blank***) | | | |
| 4 | TBI_UP_SIG_LI_2% | (Tissue = LIVER And | 98th percentile; liver; day5/7 | 0-75th percentile; | other |
| | | TimePoint >= 5 And | | liver; day5/7 | |
| | | ClinicalChemInfo = Y) | | | |
| 5 | LI_AST + ALT_INCREASE_{—} | (Tissue = LIVER And | 98th % | 25-75th % | rest |
| | >=5 hr | TimePoint >= 5 And | | | |
| | | ClinicalChemInfo = Y) | | | |
| 6 | PPAR alpha agonist_PPAR | (Tissue = LIVER) Not | (ACTIVITY_CLASS_UNION = PPAR | All else | (Zero_Class = *** |
| | alpha agonist, fibrate IN | (ACTIVITY_CLASS_UNION = *** | alpha agonist_PPAR alpha | | Blank***) |
| | LIVER | Blank***) | agonist, fibrate | | Or (Zero_Class = Y) |
| 7 | PPAR alpha agonist, fibrate | (Tissue = LIVER) Not | (STRUCTURE_ACTIVITY = PPAR | All else | (Zero_Class = *** |
| | IN LIVER | (STRUCTURE_ACTIVITY = *** | alpha agonist, fibrate | | Blank***) |
| | | Blank***) | | | Or (Zero_Class = Y) |
| 8 | HistoPredSum_LIVER_(0.25, | (TISSUE = LIVER And | Day5_LIVER-PERITONITIS SUM | Day5_LIVER- | all else |
| | 1)_LIVER- | TimePoint >= 0.25 but <= 1 And | OF SEVERITY SCORE > 0 | PERITONITIS | |
| | PERITONITIS_SUM_OF_SEVERITY > 0 | Day5_LIVER-PERITONITIS = Y) | | SUM OF | |
| | | | | SEVERITY | |
| | | | | SCORE = 0 | |
| 9 | Bile Duct Hyperplasia | (Tissue = LIVER) | 0 | 0 | 0 |
| 10 | LI_AST_INCREASE_>=5 hr | (Tissue = LIVER And | 98th % | 25-75th % | rest |
| | | TimePoint >= 5 And | | | |
| | | ClinicalChemInfo = Y) | | | |
| 11 | PXR_liver_all_HI+1_specific-1 | (Tissue = LIVER) | (PXR_Class_1_DOSE = HI) | (PXR_negative_specific = YES) | All else |
| 12 | Liver carcinogen later | (Tissue = LIVER And | Liver carcinogens and genotoxic, d3 | ALL ELSE | BLIND, |
| | timepoints | TimePoint >= 3 but <= 5) | and d5 | | AVENTIS |
| 13 | ALT, AP, and Bilirubin up | (Tissue = LIVER And | All liver REPIDS where ALT, AP, | ALL ELSE | BLIND, |
| | | TimePoint >= 3 but <= 5 And | and Bilirubin >1.5 fold increased | where ALT or | AVENTIS |
| | | ClinicalChemInfo = Y) | | AP or BIL are | |
| | | | | <1.5 | |
| 14 | ClinContDecr_LIVER_(3)_{—} | (TISSUE = LIVER And | ALBUMIN <= 5th percentile | ALBUMIN >= 35th | all else |
| | ALBUMIN_(5, 35, 0) | TimePoint = 3 And ALBUMIN = Y) | | percentile | |
| 15 | Hepatic Adenoma IN | (Tissue = LIVER) Not | (TISSUE_TOXICITY = Hepatic | All else | (Zero_Class = *** |
| | LIVER | (TISSUE_TOXICITY = *** | Adenoma) | | Blank***) |
| | | Blank***) | | | Or (Zero_Class = Y) |
| 16 | ClinContIncr_LIVER_(0.25, | (TISSUE = LIVER And | ASPARTATE | ASPARTATE | all else |
| | 1, 3, 5, 7)_ASPARTATE | TimePoint >= 0.25 but <= 7 And | AMINOTRANSFERASE >= 95th | AMINOTRANSFERASE <= 65th | |
| | AMINOTRANSFERASE_{—} | ASPARTATE | percentile | percentile | |
| | (95, 0, 65) | AMINOTRANSFERASE = Y) | | | |
| 17 | Serotonin 5-HT2B | (Tissue = LIVER) Not (IC50- | (IC50-27170\|Serotonin 5-HT2B >= −1 | (IC50- | All else |
| | DAT/NET/SERT i | 27170\|Serotonin 5-HT2B = *** | And New_Activity_Class_Unions = Monoamine | 27170\|Serotonin | |
| | | Blank***) | Re-uptake (DAT) | 5-HT2B = −3) Or | |
| | | | inhibitor_union_Monoamine Re- | (MDS_Specific_{—} | |
| | | | uptake (NET/SERT) inhibitor, | Groupings_A = 5HT_{—} | |
| | | | tricyclic_union_Monoamine Re- | agonist) | |
| | | | uptake (SERT) inhibitor, | | |
| | | | heterogeneous structures) Not | | |
| | | | (MDS_Specific_Groupings_A = 5HT_{—} | | |
| | | | agonist) | | |
| 18 | ClinContDecr_LIVER_(3, 5, | (TISSUE = LIVER And | CHOLESTEROL <= 5th percentile | CHOLESTEROL >= 35th | all else |
| | 7)_CHOLESTEROL_(5, 35, | TimePoint >= 3 but <= 7 And | | percentile | |
| | 0) | CHOLESTEROL = Y) | | | |
| 19 | H+/K+-ATPase inhibitor IN | (Tissue = LIVER And | (ACTIVITY_CLASS_UNION = H+/ | All else | (Zero_Class = *** |
| | LIVER | HighOrLowDose = HI) Not | K+-ATPase inhibitor | | Blank***) |
| | | (ACTIVITY_CLASS_UNION = *** | | | Or (Zero_Class = Y) |
| | | Blank***) | | | |
| 20 | PPAR alpha agonist IN | (Tissue = LIVER) Not | (STRUCTURE_ACTIVITY = PPAR | All else | (Zero_Class = *** |
| | LIVER | (STRUCTURE_ACTIVITY = *** | alpha agonist | | Blank***) |
| | | Blank***) | | | Or (Zero_Class = Y) |
| 21 | PXR v17 | (Tissue = LIVER) | hi dose PXR (clotrimazole, | other liver | BLIND, |
| | | | miconazole, mifepristone, | | AVENTIS LOW |
| | | | dexamethansone) KYLE | | DOSE and ALL |
| | | | | | OTHER |
| | | | | | timeponts for 1s |
| 22 | Sterol 14-demethylase | (Tissue = LIVER And | (STRUCTURE_ACTIVITY = Sterol | All else | (Zero_Class = *** |
| | inhibitor, miconazole like IN | HighOrLowDose = HI) Not | 14-demethylase inhibitor, | | Blank***) |
| | LIVER | (STRUCTURE_ACTIVITY = *** | miconazole like | | Or (Zero_Class = Y) |
| | | Blank***) | | | |
| 23 | DNA-Polymerase Inhibitor, | (Tissue = LIVER) Not | (STRUCTURE_ACTIVITY = DNA- | All else | (Zero_Class = *** |
| | thiopurine base IN LIVER | (STRUCTURE_ACTIVITY = *** | Polymerase Inhibitor, thiopurine | | Blank***) |
| | | Blank***) | base | | Or (Zero_Class = Y) |
| 24 | GABA-A agonist, non- | (Tissue = LIVER) Not | (STRUCTURE_ACTIVITY = GABA- | All else | (Zero_Class = *** |
| | NMDA-glutamate | (STRUCTURE_ACTIVITY = *** | A agonist, non-NMDA- | | Blank***) |
| | antagonist, Voltage- | Blank***) | glutamate antagonist, Voltage- | | Or (Zero_Class = Y) |
| | dependent Ca++ channel | | dependent Ca++ channel blocker, | | |
| | blocker, barbiturate IN | | barbiturate | | |
| | LIVER | | | | |
| 25 | Thyroperoxidase inhibitor | (Tissue = LIVER And | (ACTIVITY_CLASS_UNION = Thyroperoxidase | All else | (Zero_Class = *** |
| | IN LIVER | HighOrLowDose = HI) Not | inhibitor | | Blank***) |
| | | (ACTIVITY_CLASS_UNION = *** | | | Or (Zero_Class = Y) |
| | | Blank***) | | | |
| 26 | Potassium Channel [KATP] | (Tissue = LIVER) Not (IC50- | (IC50-26560\|Potassium Channel | (IC50- | All else |
| | blockers a | 26560\|Potassium Channel | [KATP] >= −1) Not | 26560\|Potassium | |
| | | [KATP] = ***Blank***) | (MDS_Specific_Groupings_B = K+_{—} | Channel [KATP] = −3) | |
| | | | channel_opener) | Or | |
| | | | | (MDS_Specific_{—} | |
| | | | | Groupings_B = K+_{—} | |
| | | | | channel_opener) | |
| 27 | ClinContIncr_LIVER_(3)_ALKALINE | (TISSUE = LIVER And | ALKALINE PHOSPHATASE >= 95th | ALKALINE | all else |
| | PHOSPHATASE_(95, 0, 65) | TimePoint = 3 And ALKALINE | percentile | PHOSPHATASE <= 65th | |
| | | PHOSPHATASE = Y) | | percentile | |
| 28 | Histamine receptor (H1) | #VALUE! | #VALUE! | #VALUE! | #VALUE! |
| | antagonist_Histamine | | | | |
| | receptor (H1) antagonist, | | | | |
| | adenosine receptor | | | | |
| | antagonist_Histamine | | | | |
| | receptor (H1) antagonist, | | | | |
| | Ca++ channel (L-Type) | | | | |
| | blocker_Histamine receptor | | | | |
| | (H1) antagonist, | | | | |
| | diphenylamine_Histamine | | | | |
| | receptor (H1) antagonist, | | | | |
| | hepatocarcinogen_{—} | | | | |
| | Histamine receptor (H1) | | | | |
| | antagonist, | | | | |
| | tricyclic_Histamine receptor | | | | |
| | (H2) antagonist_IN LIVER | | | | |
| 29 | Serotonin 5-HT2A | (Tissue = LIVER) Not (IC50- | (IC50-27165\|Serotonin 5-HT2A >= −1 | (IC50- | All else |
| | DAT/NET/SERT i | 27165\|Serotonin 5-HT2A = *** | And New_Activity_Class_Unions = Monoamine | 27165\|Serotonin | |
| | | Blank***) | Re-uptake (DAT) | 5-HT2A = −3) Or | |
| | | | inhibitor_union_Monoamine Re- | (MDS_Specific_{—} | |
| | | | uptake (NET/SERT) inhibitor, | Groupings_A = 5HT_{—} | |
| | | | tricyclic_union_Monoamine Re- | agonist) | |
| | | | uptake (SERT) inhibitor, | | |
| | | | heterogeneous structures) Not | | |
| | | | (MDS_Specific_Groupings_A = 5HT_{—} | | |
| | | | agonist) | | |
| 30 | Toxicant, heavy metal IN | (Tissue = LIVER And | (STRUCTURE_ACTIVITY = Toxicant, | All else | (Zero_Class = *** |
| | LIVER | TimePoint >= 3) Not | heavy metal | | Blank***) |
| | | (STRUCTURE_ACTIVITY = *** | | | Or (Zero_Class = Y) |
| | | Blank***) | | | |
| 31 | H2O2 radical scavenger IN | (Tissue = LIVER) Not | (ACTIVITY_CLASS_UNION = H2O2 | All else | (Zero_Class = *** |
| | LIVER | (ACTIVITY_CLASS_UNION = *** | radical scavenger | | Blank***) |
| | | Blank***) | | | Or (Zero_Class = Y) |
| 32 | Fetal Toxicity IN LIVER | (Tissue = LIVER) Not | (TISSUE_TOXICITY = Fetal | All else | (Zero_Class = *** |
| | | (TISSUE_TOXICITY = *** | Toxicity) | | Blank***) |
| | | Blank***) | | | Or (Zero_Class = Y) |
| 33 | Subcutaneous in liver later | (Tissue = LIVER And | subcutaneous administration and | ALL ELSE | BLIND, |
| | time points | TimePoint >= 3 but <= 5) | liver repid, d3 and d5 | | AVENTIS |
| 34 | PXR_liver_NoMIFE_all+1_{—} | (Tissue = LIVER) | (PXR_Class_1_all = YES) | (PXR_negative_class_{—} | All else |
| | large-1 | | | large = YES) | |
| 35 | ClinContIncr_LIVER_(5, | (TISSUE = LIVER And | ALKALINE PHOSPHATASE >= 95th | ALKALINE | all else |
| | 7)_ALKALINE | TimePoint >= 5 but <= 7 And | percentile | PHOSPHATASE <= 65th | |
| | PHOSPHATASE_(95, 0, 65) | ALKALINE PHOSPHATASE = Y) | | percentile | |
| 36 | IC50- | (Tissue = LIVER) | >=0.0000000000001 | −3 | All else |
| | 10401\|Acetylcholinesterase | | | | |
| 37 | IC50-27200\|Serotonin 5- | (Tissue = LIVER) | >−1 Not ***Blank*** | −3 | All else |
| | HT4 | | | | |
| 38 | NSAID, COX-3, | (Tissue = LIVER And | (STRUCTURE_ACTIVITY = NSAID, | All else | (Zero_Class = *** |
| | acetaminophen like IN | HighOrLowDose = HI) Not | COX-3, acetaminophen like | | Blank***) |
| | LIVER | (STRUCTURE_ACTIVITY = *** | | | Or (Zero_Class = Y) |
| | | Blank***) | | | |
| 39 | LI_CHOLESTEROL_DEC | (Tissue = LIVER And | 98th % | 25-75th % | rest |
| | REASE_>=5 hr | TimePoint >= 5 And | | | |
| | | ClinicalChemInfo = Y) | | | |

Yhe genes remaining in the dataset at the end of this stripping procedure were “depleted” for the specific classification question and could be revived only by adding back some percentage of the stripped genes ( see e. g., Example 3 below). Note that this depletion is full with respect to the selected thresold of LOR=4.0. However, this set could be depleted further if additional stripping were preformed with a second lower threshold, e.g., LOR=0.

Table 3 lists 62 of the 101 classifications where stripping resulted in a “failure” of the SPLP algprithm to produce another valid signature (LOR≧4.0) before the 4^{th} cycle of stripping. The columns in the left portion of the Table 3 with the headings “1^{st},” 2^{st},” 3^{st},” and “4^{th}, list the LOR for the best signature defined at each cycle. All 62 classification questions produced a valid gene signature at the first cycle, but only classifications 1-33 produced a valid second signature, and only classifications 1-9 produced a valid third signature. None of the 62 produced a valid fourth signature using the SPLP algorithm.

The Table 3 column labeled “sufficient set” lists the number of genes in the first and therefore “best” valid signature. The column labeled “necessary set” lists the number of genes in the union of the sufficient signatures identified each cycle with LOR≧4.00.

For the signatures 34 to 62, where failure occurred at the second cycle of computation, the necessary set is identical to the sufficient set. For signatures 10 to 33, where failure occurred at the third cycle of computation, the necessary sets correspond to the union of the genes present in the 1^{st} and 2^{nd} cycle. For the remaining 9 of the 62 signatures, the necessary set is the union of the 1^{st}, 2^{nd} and 3^{rd} cycle genes as those signatures failed at the 4^{th} cycle.

TABLE 3
62 classification questions that fail to produce a valid signature after only 4 stripping cycles

| | | Logodds ratio cycle# | | | | number of genes | |
|---|---|---|---|---|---|---|---|
| | name | 1st | 2nd | 3rd | 4th | sufficient set | necessary set |
| 1 | Monoamine Re-uptake (SERT) inhibitor, heteroge [symbol] | 5.92 | 5.29 | 4.24 | 3.87 | 79 | 311 |
| 2 | Estrogen antagonist, aromatase inhibitor IN LIVER | 4.80 | 7.10 | 4.33 | 3.84 | 49 | 170 |
| 3 | PXR_liver_NoDEX+1_specific-1_MIFE | 6.29 | 4.07 | 4.07 | 3.81 | 36 | 139 |
| 4 | DNA-alkylator IN LIVER | 6.14 | 4.49 | 4.49 | 3.72 | 68 | 234 |
| 5 | Embryotoxicity IN LIVER | 4.98 | 4.61 | 4.13 | 3.64 | 80 | 307 |
| 6 | GABAA, Benzodiazepine, timed 10 uM | 6.04 | 5.21 | 5.02 | 3.61 | 116 | 385 |
| 7 | IC50-22032\|Dopamine Transporter | 6.60 | 4.30 | 4.11 | 3.40 | 116 | 399 |
| 8 | Later timepoints CAR ligands | 5.33 | 5.03 | 4.22 | 3.32 | 62 | 199 |
| 9 | Pro-inflammatory stimuli IN LIVER | 5.03 | 4.41 | 5.75 | 1.90 | 62 | 214 |
| 10 | Testosterone_agonist c | 6.55 | 6.18 | 3.99 | | 43 | 115 |
| 11 | phospholipidosis_liver_not_fluoxetine | 5.79 | 5.12 | 3.92 | | 121 | 265 |
| 12 | Progesterone receptor agonist IN LIVER | 5.45 | 5.74 | 3.90 | | 59 | 145 |
| 13 | IC50-21460\|Calcium Channel Type L, Dihydropyr [symbol] | 4.83 | 4.39 | 3.88 | | 113 | 256 |
| 14 | IC50-17110\|Protein Serine/Threonine Kinase. ER [symbol] | 5.91 | 4.44 | 3.87 | | 99 | 231 |
| 15 | HistoCont_LIVER_(3, 5, 7)_LIVER-HEPATOCYTE | 4.83 | 4.83 | 3.83 | | 26 | 63 |
| 16 | Toxicant, free oxygen radical generator IN LIVER | 5.86 | 4.13 | 3.82 | | 120 | 292 |
| 17 | DNA damaging, free oxygen radical generator, nit [symbol] | 7.30 | 4.95 | 3.76 | | 51 | 120 |
| 18 | ALB_UP_SIG_LI_2% | 4.75 | 4.43 | 3.71 | | 43 | 90 |
| 19 | ClinSpecContDecr_LIVER_(3)_Logratio_TBI + Log | 5.85 | 4.47 | 3.70 | | 41 | 90 |
| 20 | Dopamine D1_antagonist a | 6.43 | 4.56 | 3.70 | | 114 | 240 |
| 21 | IC50-21500\|Calcium Channel Type L, Phenylalkyl [symbol] | 5.53 | 4.38 | 3.67 | | 114 | 247 |
| 22 | DNA damaging, free oxygen radical generator IN [symbol] | 5.25 | 4.43 | 3.67 | | 86 | 213 |
| 23 | Estrogen ERalpha_antagonist a | 6.58 | 5.07 | 3.61 | | 67 | 154 |
| 24 | Bacterial ribosomal (50S) function inhibitor, macro | 4.63 | 4.67 | 3.56 | | 66 | 146 |
| 25 | Dopamine receptor antagonist (D), phenothiazine I | 4.67 | 4.10 | 3.55 | | 136 | 301 |
| 26 | Estrogen antagonist, aromatase inhibitor_Estroger | 5.67 | 4.27 | 3.41 | | 90 | 211 |
| 27 | Ca++ channel (L-Type) blocker_Ca++ channel (L- | 5.57 | 4.39 | 3.36 | | 83 | 193 |
| 28 | HistoCont_LIVER_(5, 7)_LIVER-FATTY CHANGE | 4.83 | 6.24 | 3.36 | | 38 | 87 |
| 29 | Sterol 14-demethylase inhibitor_Sterol 14-demeth [symbol] | 4.62 | 4.35 | 3.32 | | 106 | 234 |
| 30 | AP_UP_SIG_LI_2%_B | 4.58 | 4.04 | 3.27 | | 20 | 50 |
| 31 | ClinPredDecr_LIVER_(0.25)_LIPASE_(5, 35, 65) | 6.78 | 5.79 | 2.79 | | 28 | 62 |
| 32 | LI_HEMOGLOBIN_DECREASE_>=5 hr | 6.04 | 5.18 | 2.77 | | 28 | 61 |
| 33 | HistoPredSum_LIVER_(0.25, 1)_LIVER-NECROS | 5.18 | 4.09 | 0.00 | | 38 | 100 |
| 34 | 5HT2/D4/D2 antagonist, tricyclic antipsychotic_5H | 6.52 | 0.00 | | | 30 | 30 |
| 35 | IC50-21755\|Chemokine CCR2B | 5.59 | 2.81 | | | 71 | 71 |
| 36 | LI_HEMATOCRIT_INCREASE_>=5 hr | 5.58 | 3.90 | | | 26 | 26 |
| 37 | NSAID, COX-2/1, coxib like IN LIVER | 5.55 | 3.40 | | | 59 | 59 |
| 38 | Hepatocellular Carcinoma IN LIVER | 5.45 | 0.00 | | | 57 | 57 |
| 39 | NSAID, COX-1_NSAID, COX-1, 6-Methoxy-napht [symbol] | 5.30 | 3.90 | | | 85 | 85 |
| 40 | IC50-28501\|Testosterone | 5.25 | 3.93 | | | 163 | 163 |
| 41 | GABA-A agonist, benzodiazepin, long acting IN LI [symbol] | 5.12 | 1.68 | | | 38 | 38 |
| 42 | IC50-26011\|Opiate delta | 5.09 | 3.87 | | | 135 | 135 |
| 43 | REL_LIVER_WT_UP_SIG_LI_2% | 5.06 | 3.93 | | | 29 | 29 |
| 44 | HistoPred_LIVER_(0.25, 1)_LIVER-NECROSIS_( [symbol] | 5.05 | 2.46 | | | 34 | 34 |
| 45 | ClinContDecr_LIVER_(3, 5, 7)_LYMPHOCYTE_(5 | 4.93 | 3.88 | | | 71 | 71 |
| 46 | IC50-27191\|Serotonin 5-HT3 | 4.74 | 3.14 | | | 106 | 106 |
| 47 | IC50-20420\|Adrenergic beta3 | 4.73 | 3.99 | | | 140 | 140 |
| 48 | Bacterial ribosomal (30S) function inhibitor, tetrac [symbol] | 4.71 | 0.00 | | | 57 | 57 |
| 49 | IC50-27820\|Sigma2 | 4.68 | 3.74 | | | 138 | 138 |
| 50 | ClinContDecr_LIVER_(3, 5, 7)_LEUKOCYTE CO [symbol] | 4.65 | 3.33 | | | 88 | 88 |
| 51 | Estrogen receptor agonist, environmental toxicant | 4.65 | 2.98 | | | 127 | 127 |
| 52 | IC50-27951\|Sodium Channel, Site 2 | 4.61 | 3.43 | | | 89 | 89 |
| 53 | Muscarinic M2_antagonists e | 4.56 | 3.11 | | | 153 | 153 |
| 54 | PXR_liver_all_HI+1_ligand-1 | 4.54 | 3.83 | | | 26 | 26 |
| 55 | Bacterial folate synthesis inhibitor, dihydropteroate | 4.49 | 3.78 | | | 51 | 51 |
| 56 | Estrogen ERalpha_agonist d | 4.46 | 3.52 | | | 168 | 168 |
| 57 | IC50-20051\|Adenosine A1 | 4.38 | 3.13 | | | 100 | 100 |
| 58 | ClinSpecContIncr_LIVER_(0.25, 1, 3, 5, 7)_Lograt | 4.33 | 3.69 | | | 115 | 115 |
| 59 | IC50-19401\|Thromboxane Synthase | 4.25 | 3.63 | | | 136 | 136 |
| 60 | LI_LEUKOCYTE_COUNT_INCREASE on Day5_0 | 4.15 | 3.47 | | | 58 | 58 |
| 61 | ClinContIncr_LIVER_(5, 7)_ABSOLUTE SEGMEN [symbol] | 4.13 | 3.14 | | | 27 | 27 |
| 62 | LI_CREATININE_INCREASE_5 | 4.02 | 2.75 | | | 51 | 51 |

Table 4 lists the specific 79 genes of the monoamine re-uptake (SERT) inhibitor ature (i.e., classification 1 from Table 2 above) after the first cycle. Each of the 79 genes is ed with its corresponding weight. A bias of 1.69 was used in deriving the weights.

TABLE 4

| Gene | Weight |
|---|---|
| AI103937 | 1.39 |
| NM_019123 | 0.88 |
| AW141940 | 0.79 |
| X78604 | 0.75 |
| AW914758 | 0.64 |
| AI639012 | 0.51 |
| NM_017288 | 0.42 |
| AA944403 | 0.41 |
| AF171936 | 0.41 |
| AI069922 | 0.37 |
| AA893164 | 0.37 |
| NM_019292 | 0.36 |
| AI144644 | 0.35 |
| AI070137 | 0.33 |
| AW915662 | 0.33 |
| AF187814 | 0.32 |
| AW918740 | 0.28 |
| U42975 | 0.27 |
| M84203 | 0.25 |
| AA924151 | 0.24 |
| AI412889 | 0.22 |
| AF054826 | 0.22 |
| BF405468 | 0.21 |
| U46118 | 0.21 |
| D13962 | 0.16 |
| BF558694 | 0.12 |
| U08136 | 0.1 |
| M35495 | 0.09 |
| AW531530 | 0.08 |
| AF001896 | 0.08 |
| AF098301 | 0.08 |
| AB018546 | 0.06 |
| U71294 | 0.06 |
| AI407409 | 0.06 |
| BF407531 | 0.05 |
| BE095840 | 0.05 |
| AF045564 | 0.05 |
| NM_017099 | 0.03 |
| U10188 | −0.03 |
| BF413176 | −0.04 |
| AI179459 | −0.04 |
| AA891221 | −0.04 |
| D14819 | −0.04 |
| BG153368 | −0.05 |
| AI409738 | −0.06 |
| BE109513 | −0.07 |
| AF027331 | −0.08 |
| AA894030 | −0.08 |
| BF522317 | −0.09 |
| BF411727 | −0.11 |
| NM_013068 | −0.12 |
| BE104931 | −0.12 |
| AW143082 | −0.13 |
| BF551118 | −0.13 |
| D79981 | −0.14 |
| AW917712 | −0.14 |
| AI227742 | −0.17 |
| NM_012521 | −0.17 |
| AI407719 | −0.17 |
| AI228598 | −0.19 |
| AI234719 | −0.22 |
| AW142280 | −0.22 |
| AI233740 | −0.22 |
| BF557691 | −0.26 |
| BE114586 | −0.27 |
| U04319 | −0.3 |
| AI410352 | −0.33 |
| NM_012875 | −0.36 |
| AI172175 | −0.37 |
| AF182946 | −0.37 |
| AI179711 | −0.42 |
| AI169591 | −0.42 |
| NM_021848 | −0.51 |
| D29969 | −0.61 |
| BF282574 | −0.71 |
| BF282370 | −0.72 |
| BE119802 | −0.91 |
| AI010033 | −1.11 |
| AI236054 | −1.83 |
| Bias | 1.69 |

Table 5 lists the 311 genes in the necessary set of the monoamine re-uptake (SERT) inhibitor signature derived according to the stripping method described above. In performing the stripping both the first and second LOR threshold value were set at greater than or equal to 4.0. The necessary set represents the union of the genes in the signatures derived in the 1^{st}, 2^{nd}, and 3^{rd} stripping cycles shown above in Table 3.

TABLE 5

| Gene | Gene | Gene | Gene |
|---|---|---|---|
| AI103937 | AI639012 | AA893164 | AF187814 |
| NM_019123 | NM_017288 | NM_019292 | AW918740 |
| AW141940 | AA944403 | AI144644 | U42975 |
| X78604 | AF171936 | AI070137 | M84203 |
| AW914758 | AI069922 | AW915662 | AA924151 |
| AI412889 | AI234719 | AW915682 | AW917460 |
| AF054826 | AW142280 | NM_019147 | NM_021701 |
| BF405468 | AI233740 | AI007936 | AI716417 |
| U46118 | BF557691 | D83044 | U66292 |
| D13962 | BE114586 | BE112237 | AW916860 |
| BF558694 | U04319 | D10693 | BF549441 |
| U08136 | AI410352 | NM_017261 | AW434092 |
| M35495 | NM_012875 | NM_019905 | U41662 |
| AW531530 | AI172175 | AI410438 | AB026288 |
| AF001896 | AF182946 | AA924717 | L05435 |
| AF098301 | AI179711 | M35106 | BF398716 |
| AB018546 | AI169591 | AI172165 | AW915749 |
| U71294 | NM_021848 | NM_019306 | BF557299 |
| AI407409 | D29969 | M34643 | AB009636 |
| BF407531 | BF282574 | AI008125 | BE108235 |
| BE095840 | BF282370 | AF022247 | X59290 |
| AF045564 | BE119802 | NM_013197 | NM_012704 |
| NM_017099 | AI010033 | NM_021858 | BE111699 |
| U10188 | AI236054 | AI410096 | M13979 |
| BF413176 | AA945696 | BE113060 | AI178784 |
| AI179459 | AW916308 | BF551377 | AF132046 |
| AA891221 | NM_019180 | X63574 | AI236618 |
| D14819 | BE095474 | U41853 | BF281133 |
| BG153368 | AI103988 | AA942695 | AF110026 |
| AI409738 | AA858518 | J04486 | BE107051 |
| BE109513 | AI058938 | Y00697 | U27518 |
| AF027331 | NM_013070 | AF041838 | D85435 |
| AA894030 | BF281544 | AI170783 | BE111634 |
| BF522317 | NM_021759 | AW917572 | AW919837 |
| BF411727 | D13555 | BF405086 | BF419628 |
| NM_013068 | AW917160 | AF106659 | BF524978 |
| BE104931 | BE113423 | AF117820 | AW919982 |
| AW143082 | D10763 | AB013732 | M83560 |
| BF551118 | BE102266 | BF394166 | AI105205 |
| D79981 | AF081582 | BF394170 | AW918222 |
| AW917712 | U92010 | NM_012834 | AW918431 |
| AI227742 | AA944526 | BF405917 | BF551345 |
| NM_012521 | BE113316 | AI232205 | AI407113 |
| AI407719 | AI172266 | BE101094 | AW919429 |
| AI228598 | AA850725 | BE108249 | AI711305 |
| AW531902 | U25281 | M73486 | AW144684 |
| AI599479 | NM_012699 | BF394563 | NM_012869 |
| BE095664 | NM_013034 | AI411412 | M33936 |
| AI233729 | NM_021774 | AW534166 | AI169377 |
| AI411391 | AI179460 | X78949 | AI412967 |
| AI178818 | AF271156 | D14839 | BF556836 |
| AI229529 | BE101274 | U67914 | AW919239 |
| M25073 | AI176548 | AI007985 | BE105305 |
| AI013800 | AF151367 | AA818197 | AJ222691 |
| BE098799 | NM_021585 | NM_013075 | AI176792 |
| AI230988 | AW915643 | AA891839 | AA850909 |
| AA899898 | NM_012903 | AF021923 | D90036 |
| AW916920 | BE113268 | X56228 | BF284803 |
| AW143513 | U31866 | AI413058 | BF397951 |
| BE113340 | AI169225 | D78482 | BE118454 |
| NM_017110 | NM_012707 | AW920343 | AI502229 |
| AI177412 | AB046606 | AI231808 | AW530773 |
| BF395101 | NM_019280 | AB021980 | AF061947 |
| AA851386 | AI072459 | AI716265 | L36388 |
| AW914808 | AF037071 | BE107128 | BE095971 |
| AI598507 | AJ132230 | AI178768 | BF408841 |
| AI102026 | BF392959 | NM_013133 | AI407992 |
| AF071501 | L36459 | AA875129 | AI176477 |
| AI407187 | BF522695 | NM_013215 | NM_020471 |
| X06564 | NM_012578 | AI406885 | AI406487 |
| BE101480 | AI011505 | AI071187 | AI011716 |
| BF399614 | BE111710 | AI716471 | AI009644 |
| L09752 | NM_012955 | L36088 | AA901066 |
| AA851369 | AI104125 | AI012498 | AI237657 |
| NM_017175 | AI169629 | NM_017180 | AI010312 |
| NM_012497 | AF057564 | NM_013217 | BF282686 |
| AW142852 | BF549650 | AW918478 | AW917069 |
| AI145359 | BF400832 | AF021854 | |

Table 6 lists the remaining 39 of the 101 liver-based chemogenomic classifications where stripping did not result in failure of the SPLP algorithm to identify a valid signature even after 4 cycles. As in Table 3, the column labeled “sufficient set” lists the number of genes in the initial “best” sufficient signature. The column labeled “necessary set” lists the number of genes in the union of sufficient signatures identified at each of the four cycles. Because all of the 39 classifications produced a valid signature even after 4 cycles, the number in the “necessary set” column represents the minimum number in the necessary set for that classification question.

TABLE 6
39 classification questions that continue to produce valid signatures even after 4 stripping cycles

| | | Logodds ratio cycle# | | | | number of genes | |
|---|---|---|---|---|---|---|---|
| | name | 1st | 2nd | 3rd | 4th | sufficient set | necessary set |
| 1 | HMG-CoA reductase inhibitors IN LIVER | 10.03 | 7.19 | 9.26 | 7.48 | 15 | >86 |
| 2 | Estrogen receptor agonist_Estrogen receptor agonist, steroidal IN LIVE [symbol] | 10.28 | 9.27 | 6.12 | 6.92 | 36 | >139 |
| 3 | Estrogen receptor antagonist/agonist, tissue specific IN LIVER | 8.73 | 7.74 | 6.89 | 6.89 | 37 | >181 |
| 4 | TBI_UP_SIG_LI_2% | 6.44 | 6.88 | 6.88 | 6.88 | 15 | >67 |
| 5 | LI_AST+ALT_INCREASE_>=5 hr | 6.39 | 6.82 | 6.82 | 6.82 | 17 | >56 |
| 6 | PPAR alpha agonist_PPAR alpha agonist, fibrate IN LIVER | 11.39 | 8.96 | 7.44 | 6.77 | 52 | >200 |
| 7 | PPAR alpha agonist, fibrate IN LIVER | 7.50 | 7.19 | 7.07 | 6.25 | 40 | >165 |
| 8 | HistoPredSum_LIVER_(0.25, 1)_LIVER- | 6.92 | 4.40 | 6.19 | 6.19 | 31 | >131 |
| | PERITONITIS_SUM_OF_SEV [symbol] | | | | | | |
| 9 | Bile Duct Hyperplasia | 9.24 | 8.81 | 8.36 | 6.06 | 32 | >142 |
| 10 | LI_AST_INCREASE_>=5 hr | 6.43 | 5.48 | 5.99 | 5.99 | 14 | >49 |
| 11 | PXR_liver_all_HI+1_specific-1 | 11.20 | 8.34 | 5.13 | 5.98 | 18 | >81 |
| 12 | Liver carcinogen later timepoints | 9.04 | 8.00 | 5.97 | 5.75 | 41 | >171 |
| 13 | ALT, AP, and Bilirubin up | 6.33 | 5.71 | 6.07 | 5.45 | 22 | >89 |
| 14 | ClinContDecr_LIVER_(3)_ALBUMIN_(5, 35, 0) | 5.73 | 7.35 | 5.57 | 5.40 | 34 | >130 |
| 15 | Hepatic Adenoma IN LIVER | 7.06 | 6.19 | 5.19 | 5.40 | 55 | >208 |
| 16 | ClinContIncr_LIVER_(0.25, 1, 3, 5, 7)_ASPARTATE AMINOTRANSFE [symbol] | 7.56 | 6.42 | 5.41 | 5.36 | 46 | >192 |
| 17 | Serotonin 5-HT2B DAT/NET/SERT i | 8.00 | 5.92 | 5.16 | 5.16 | 78 | >330 |
| 18 | ClinContDecr_LIVER_(3, 5, 7)_CHOLESTEROL_(5, 35, 0) | 8.56 | 5.78 | 5.42 | 5.10 | 53 | >215 |
| 19 | H+/K+-ATPase inhibitor IN LIVER | 7.52 | 6.07 | 5.78 | 5.01 | 42 | >187 |
| 20 | PPAR alpha agonist IN LIVER | 7.55 | 7.18 | 4.34 | 5.00 | 63 | >232 |
| 21 | PXR v17 | 7.28 | 6.82 | 5.54 | 4.94 | 28 | >110 |
| 22 | Sterol 14-demethylase inhibitor, miconazole like IN LIVER | 5.86 | 6.45 | 4.87 | 4.87 | 53 | >223 |
| 23 | DNA-Polymerase Inhibitor, thiopurine base IN LIVER | 8.37 | 4.95 | 8.06 | 4.79 | 123 | >410 |
| 24 | GABA-A agonist, non-NMDA-glutamate antagonist, Voltage-dependent | 5.63 | 4.79 | 5.11 | 4.79 | 64 | >245 |
| 25 | Thyroperoxidase inhibitor IN LIVER | 6.85 | 4.64 | 4.64 | 4.64 | 33 | >135 |
| 26 | Potassium Channel [KATP] blockers a | 5.67 | 4.95 | 4.87 | 4.50 | 48 | >200 |
| 27 | ClinContIncr_LIVER_(3)_ALKALINE PHOSPHATASE_(95, 0, 65) | 5.05 | 4.46 | 4.18 | 4.46 | 45 | >189 |
| 28 | Histamine receptor (H1) antagonist_Histamine receptor (H1) antagonis | 4.43 | 4.43 | 4.06 | 4.43 | 57 | >271 |
| 29 | Serotonin 5-HT2A DAT/NET/SERT i | 7.89 | 7.42 | 6.11 | 4.31 | 50 | >185 |
| 30 | Toxicant, heavy metal IN LIVER | 4.75 | 4.16 | 4.16 | 4.30 | 55 | >200 |
| 31 | H2O2 radical scavenger IN LIVER | 6.66 | 4.09 | 4.09 | 4.28 | 74 | >280 |
| 32 | Fetal Toxicity IN LIVER | 7.22 | 5.87 | 5.03 | 4.22 | 58 | >267 |
| 33 | Subcutaneous in liver later time points | 5.93 | 4.89 | 4.69 | 4.18 | 92 | >351 |
| 34 | PXR_liver_NoMIFE_all+1_large-1 | 6.16 | 5.12 | 4.81 | 4.18 | 43 | >178 |
| 35 | ClinContIncr_LIVER_(5, 7)_ALKALINE PHOSPHATASE_(95, 0, 65) | 6.13 | 4.64 | 4.29 | 4.18 | 27 | >127 |
| 36 | IC50-10401\|Acetylcholinesterase | 7.74 | 5.96 | 4.36 | 4.14 | 72 | >278 |
| 37 | IC50-27200\|Serotonin 5-HT4 | 6.50 | 5.28 | 4.79 | 4.12 | 49 | >208 |
| 38 | NSAID, COX-3, acetaminophen like IN LIVER | 5.18 | 4.33 | 5.32 | 4.06 | 65 | >255 |
| 39 | LI_CHOLESTEROL_DECREASE_>=5 hr | 5.17 | 4.79 | 4.32 | 4.05 | 21 | >70 |

The results depicted in Table 3 indicate that for many gene expression based signatures (e.g., 62 out of 101), 1-3 valid non-overlapping gene signatures may be generated and consequently, the necessary set is just 2-3 times larger than the sufficient set of variables. The results shown in Table 6, however, demonstrate that a substantial number of classification questions generate a large number of non-overlapping valid signatures. In those cases, the necessary set must be on average at least four-fold larger than the best sufficient set.

In order to confirm these results and to determine the size of the necessary set for some of the more degenerate classification tasks, one classification question that failed at the 2^{nd} cycle (NSAID, cox2/1,coxib like) and three classification questions that did not fail even up to the 4^{th} cycle (HMG CoA Reductase, Bile Duct Hyperplasia, PPARα) were analyzed in greater depth. Specifically, the procedure outlined above was repeated but the algorithm was allowed to proceed until all LOR drop below 4.0.

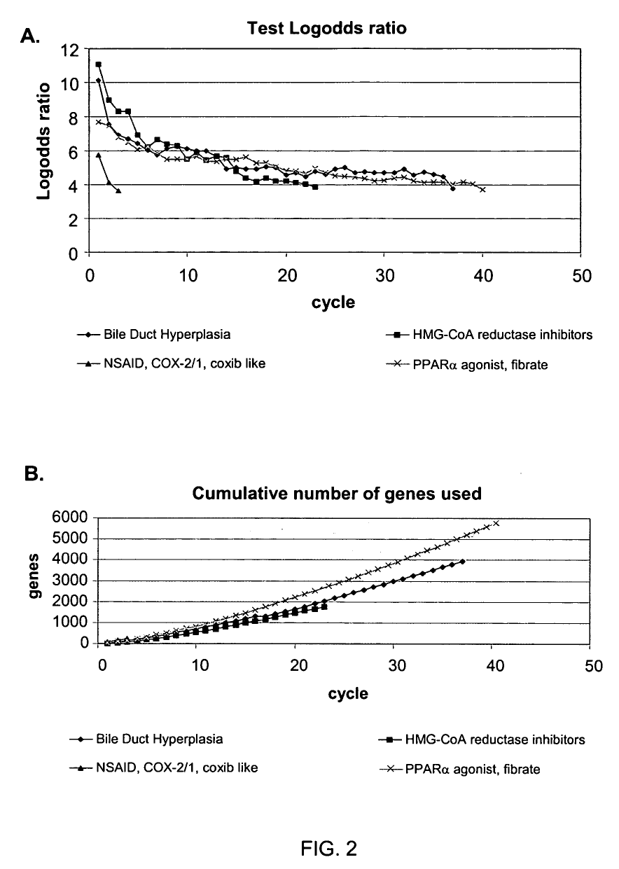

As shown by the plot depicted in FIG. 2A, the “NSAID,cox2/1,coxib like” classification question rapidly failed at the third cycle of stripping, whereas the other three did not fail (i.e., no signature with LOR≧4.00) until much later. HMG CoA Reductase, bile duct hyperplasia and PPARα classifications only failed at the 23^{rd}, 37^{th} and 40^{th} cycle respectively, yielding necessary sets of 1771, 3937 and 5706 genes, respectively (see FIG. 2B). It should be noted that if the threshold for a valid signature is set at LOR=6.0, the HMG CoA Reductase, bile duct hyperplasia and PPARα classifications each fail at about the seventh cycle, and consequently, the necessary set for each is reduced to about 300-500 genes.

### EXAMPLE 3

This example illustrates how the necessary set of genes for a classification question may be functionally characterized by randomly supplementing and thereby restoring the ability of a depleted dataset to generate signatures above an average LOR. In addition to demonstrating the power of the information rich genes in a necessary set, this example illustrates a system for describing any necessary set of genes in terms of its performance parameters.

As described in Example 2, a necessary set of 311 genes (see Table 5) for the SERT inhibitor classification question was generated via the stripping method. In the process, a corresponding fully depleted set of 8254 genes (i.e., the full dataset of 8565 genes minus 311 genes) was also generated. The fully depleted set of 8254 genes was not able to generate a SERT inhibitor signature capable of performing with a LOR greater than or equal to 4.00.

A further 311 genes were randomly removed from the fully depleted set. Then a randomly selected set including 5, 10, 20, 40 or 80% of the genes from either: (a) the necessary set; or (b) the set of 311 randomly removed from the fully depleted set; were added back to the depleted set minus 311. The resulting “supplemented depleted” set was then used to generate a SERT inhibitor signature, and the performance of this signature was cross-validated. This process was repeated 50 times each for the depleted set supplemented with some percentage of genes from the necessary set and supplemented with the random 311 genes removed from the original depleted set. Fifty cross-validated SERT inhibitor signatures were obtained for each various percentages of depleted set supplementation. Average LOR values were calculated based on the 50 signatures generated in each case.

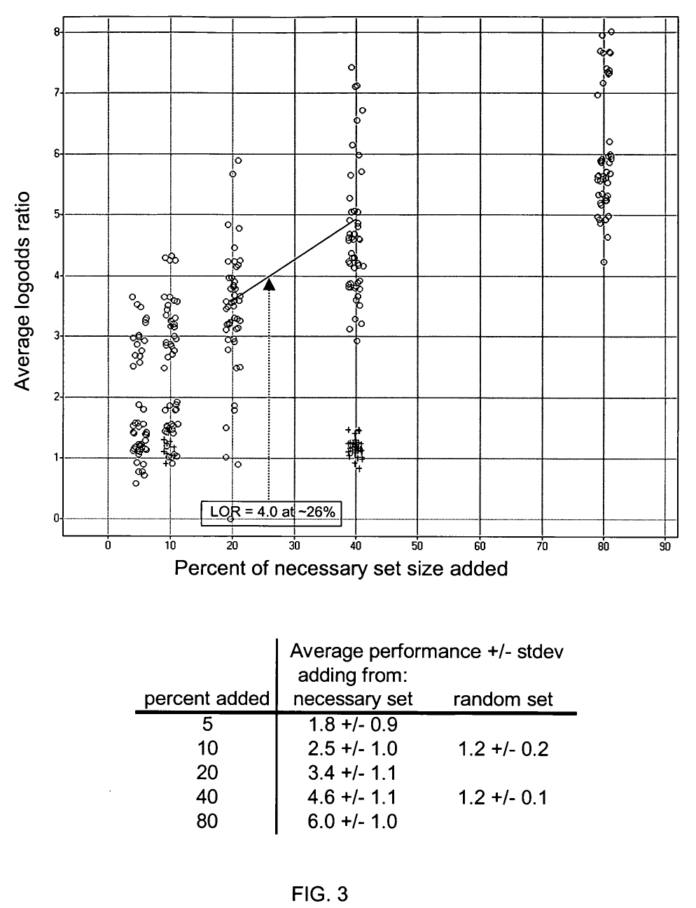

The power of the information rich genes in the necessary set was demonstrated by the results tabulated in FIG. 3. Supplementing the fully depleted set (minus random 311) with as few as 5% of the randomly chosen genes from the necessary set resulted in significantly improved performance (i.e., increase from avg. LOR=1.2 to 1.8). In contrast, supplementing the depleted set (minus random 311) with 10%, or even 40% of the random 311 genes to failed to cause any improvement in performance (LOR remains 1.2) for generating SERT inhibitor signatures.

The above shows how supplementation with necessary set genes “revives” a fully depleted set. This ability is a common characteristic of any necessary set. This functional characteristic may be quantified with a plot of avg. LOR versus the percentage of random genes used to supplement the depleted set. As shown by the plot in FIG. 3, for the SERT signature it was found that 26% of the necessary set of 311 genes restores an avg. LOR=4.0 to the fully depleted set whose performance is LOR ˜1.2. Thus, the necessary set of genes may be functionally characterized as the set of genes for which a randomly selected 26% will supplement a fully depleted set with avg. LOR ˜1.2, such that the resulting set performs with an average LOR greater than or equal to 4.00.

### EXAMPLE 4

This example illustrates how the stripping method of Example 2 may be used to carry out a functional analysis of genes within the non-overlapping sufficient signature of the PPARα necessary set.

All of the valid classifiers for a given classification question must by definition overlap with the necessary gene set as defined herein. This is a direct consequence of the fact that the fully depleted set (the remaining genes after the last successful cycle of stripping) cannot produce a valid classifier. It should be informative to submit the necessary set to functional analysis because this gene set constitutes all the genes that in some combination can yield a valid classifier for a specific classification question.

Clustering Analysis Offirst Five Sufficient Sets

A preliminary analysis was performed of the 317 genes identified in the first 5 cycles of the PPARα signature stripping procedure. Starting with a table (genes are rows and compound treatments are columns) of gene expression logratios, a table of the weighted expression (also referred to as the gene's “impact”) was produced where each line, corresponding to a gene, was multiplied by its weight in the corresponding signature. The vertical dimension of the table was reduced by generating a single column for the maximum weighted expression (impact) achieved by a drug under any treatment conditions. Most drugs were tested at two doses and four time points. This procedure thus reduces the number of columns by a factor of eight.

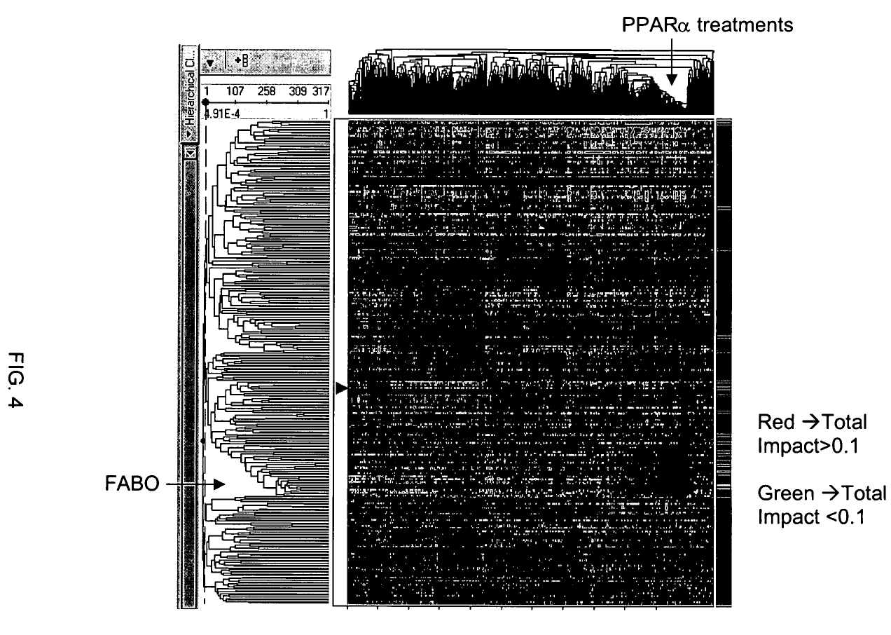

The weighted table was clustered using UPGMA, a standard algorithm available through Spotfire DecisionSite™ to produce the image depicted in FIG. 4. The coloring scheme was set to green for negative gene impact values and red for positive gene impact values. According to the scalar product decision rule described above, positive weighted values for a gene in a given treatment tend to assign this treatment to the class of interest (PPARα in this case) while negative values tend to pull away from the class. One can further summarize the behavior of a specific gene by summing its impact across all compound treatments. The scale of these overall summed impacts is depicted by the column of colored bars to the right in FIG. 4. A large positive value for the overall impact sum indicated that the gene in question acts on average as a reward for the class of interest while a negative value indicates that the gene acts on average as a penalty.

FIG. 4 shows a single major “dip” in both the clustered tree of compound treatments (x-axis) and in the clustered tree of genes (y-axis). The dip in the clustered tree of compound treatments corresponded mostly to PPARα agonists; this is expected since the PPARα signature is a two class classifier for that group of treatments. The single dip in the gene tree corresponds mostly to the fatty acid beta oxidation genes (FABO). This branch also corresponds to where most of the reward genes are located (marked in red in the rightmost column). This result suggests that during the initial cycles of stripping the algorithm is using mostly FABO genes as reward genes.

PPARα agonists induce FABO genes (see e.g., Kersten, S., B. Desvergne, and W. Wahli, “Roles of PPARs in health and disease,” Nature 405: 421-424 (2000)), and FABO genes are used as reward genes in the initial signature run (see e.g., Natsoulis et al. 2004, Gen. Res.). This result suggests that after five cycles of stripping the algorithm keeps replacing the eliminated FABO reward genes with other FABO genes to produce a valid classifier. The rightmost column of FIG. 4 also shows that only a minority of the genes act as reward genes most others are penalty genes. Generally, penalty genes do not tend to form tight clusters.

Non-Overlapping Signatures can be used to Confirm Signature Hits

The stripping procedure described above may be used to confirm signature hits. For example, it was previously observed that an unknown compound (“compound X”) had a positive scalar product when analyzed against the PPARα signature, however the scalar product was near that of the weakest of the known PPARα agonists, clofibric acid. In this situation, the question arises whether compound X is a “false” positive hit. For example, the apparent match of compound X to the PPARα signature may have been the result of an artifact on the expression microarray that escaped quality control. Given that each successive signature obtained by stripping is composed of a different set of genes (or at least a different set of probes on the array) these independently derived signatures may be used to confirm the match of an unknown to a signature.

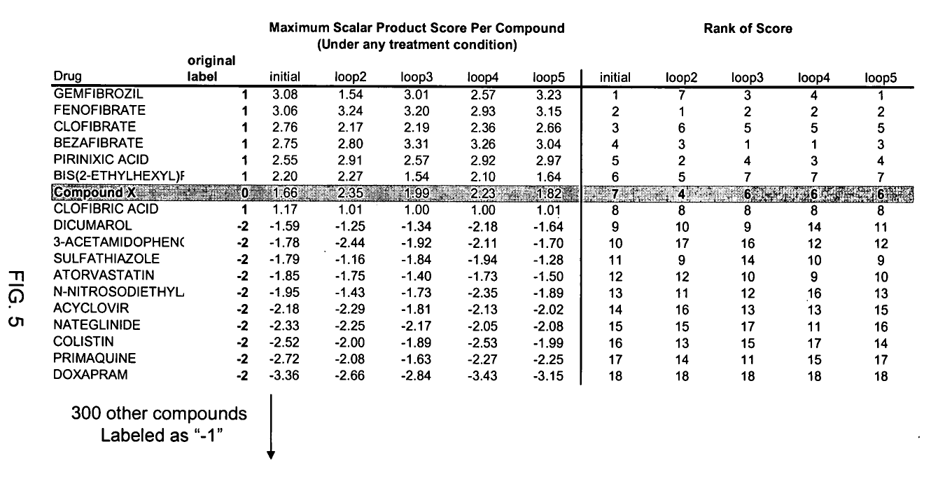

To illustrate this application the PPARα label set was modified. Originally, the unmodified labels for the PPARα signature were set such that all known PPARα agonists (42 treatments corresponding to 8 compounds) were labeled as “+1” and all treatments (˜1600) with other drugs (˜310) were labeled as “−1”. These PPARα label set was modified as follows: 10 randomly chosen non-PPARα compounds were set aside and not used in the generation of a new PPARα signature. These set aside compound treatment experiments were labeled “−2” to distinguish them from the unknown compound treatment which, was labeled as “0”. Neither the “0” labeled not the “−2” labeled compounds take part in the signature generation. The new PPARα signature was trained for the 8 known PPARα compounds (labeled “+1”) against the other 300 non-PPARα compounds (labeled as “−1”). The maximum scalar product achieved under any treatment condition was calculated for each compound and for each of the 5 cycles of stripping. As shown by the results tabulated in FIG. 5, compound X consistently scored a scalar product >1 regardless of the stripping cycle (i.e., “loop” 1-5). It is ranked above the 10 set-aside compounds and close to the rank of clofibric acid. This consistent score with five different signatures confirms that compound X is a member of the PPARα antagonist class. The consistently low value of its scalar product also places compound X close to clofibric acid as a weak member of the PPARα class.

GO Analysis of PPARα Gene Sets

The complete results for the PPARα signature show that 40 cycles of stripping, involving 5706 genes, were needed to define the necessary set for this signature. A repeat of the analysis described in FIG. 4 on the complete results shows that only 234 of the 5706 genes are reward genes. The 234 reward genes were submitted to GO (Gene Ontology) statistical analysis.

The hypergeometric formula was used to assess the significance of the enriched GO terms. The most significantly enriched GO term in the 234 reward genes is unsurprisingly FABO and several other terms related to lipid metabolism. All metabolism genes were subtracted from the set of 234 reward genes and the remaining set was submitted again to the same analysis. The most significant term in this second analysis was “transport.” A third round of analysis revealed “adhesion” as the most significant term. No other significant terms were detected after subtracting adhesion related genes.

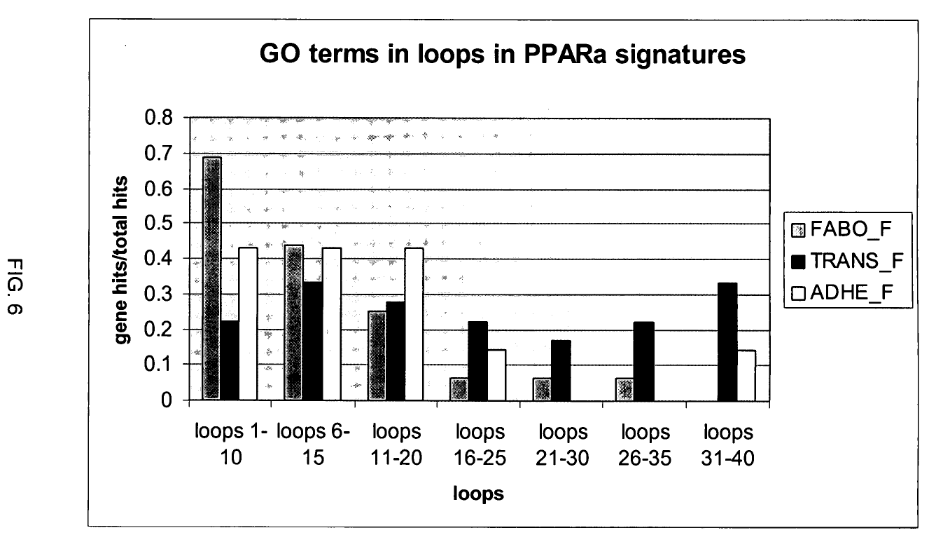

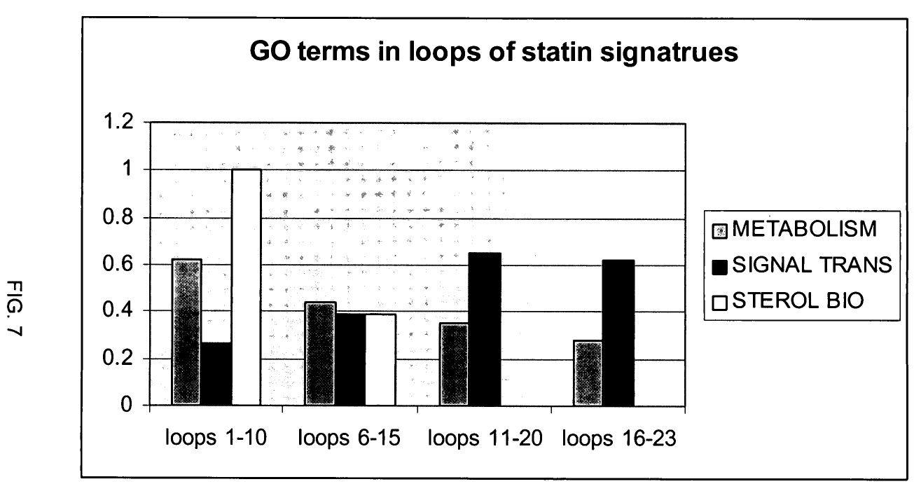

In order to determine whether genes belonging to these three GO terms are used successively the enrichment in each of the three terms was plotted as a function of the cycles (referred to in FIGS. 6 and 7 as “loops”) in which they appear. FIG. 6 shows that the first genes to be used are the FABO genes as suggested by the clustering analysis illustrated in FIG. 4. The use of FABO genes decreases regularly, falling to a low level by cycle 15 and disappearing altogether by cycle 30. Adhesion related genes become the most prominent group by cycle 16. The use of adhesion-related genes subsequently decreases. An intermediate level of transport is used throughout the 40 cycles.

Identification of an Alternate Pathway Correlation for PPARα Agonists.

The fact that adhesion and transport genes may be used to classify the effect induced by PPARα agonists indicates that these genes may be targets for PPARα related diseases. These alternate PPARα related genes are believed to be novel and unlikely to be uncovered by other functional analysis methods in large part because of the predominant effect of the FABO genes. Uncovering alternate pathways whose gene expression is altered in a characteristic manner by PPARα agonists may have great biological significance. While the PPARα agonists are known to induce beta oxidation they are also known to induce peroxisomal proliferation, at least in rodents, and peroxisomal proliferation may be the cause of the increased liver cancers observed in rodents exposed to PPARα agonists. PPARα agonists do not cause peroxisomal proliferation in humans, yet the suspicion remains that they may still elevate the risks of liver cancer.

Thus, the present analysis reveals a plurality of distinct gene signatures, all of them sufficient to classify of the effect of PPARα agonists as they meet the LOR≧4.0 threshold criteria for signature validation. By design, none of these signatures overlap by a single gene. Yet the stripping algorithm reveals that the signatures tend to use initially the induction of some of the more prominent and well recognized FABO genes while they only later use other pathways such as adhesion and transport. The signatures using predominantly adhesion molecules may be used as a marker for important side effects of PPARα agonists in rodents. The same genes or their orthologs could also form the basis of a diagnostic to detect early signs of neoplastic transformation in liver biopsies of PPARα agonist treated humans.

### EXAMPLE 5

Functional Analysis of the Non-Overlapping Sufficient Sets Within the HMG CoA (statin) Necessary Set

A similar functional analysis of the HMG CoA Reductase (statin) signatures may be carried out according to the methods described in Example 4. The HMG CoA Reductase (statin) signatures revealed by the stripping algorithm defined a necessary gene set composed of 1771 genes. Of these 168 are reward genes. The GO analysis described above for the PPARα signature was repeated for the statin signature. The most significant GO term in the set of reward genes is “sterol metabolism.” This result is not surprising as statins are known to induce many cholesterol biosynthesis genes. Removing “metabolism,” a superset of the “sterol metabolism” genes, reveals that signal transduction genes constitute the next most significant term.

The enrichment of the three terms (sterol metabolism, metabolism and signal transduction) was graphed as function of stripping cycles (FIG. 7). It is apparent for this graph that sterol metabolism is used first and signal transduction is used later. Again, as shown above for the PPARα agonist class of drugs, this stripping analysis appears to reveal valuable independent biomarkers for the secondary effects of statin drugs.

Recently substantial effort has been devoted to the study of the multiple therapeutically beneficial effects of statin drugs. The direct effects of statins on cholesterol biosynthesis are well-known. The recognition that statins may have anti-proliferative and anti-inflammatory properties, both of which may contribute to the control of atherosclerosis, has only recently been suggested. The above-described analysis of the necessary set of genes relevant to statin classifiers provides further support for this new hypothesis.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

What is claimed:

1. A method for determining the necessary set of variables for a classification question, said method comprising:
a. deriving a first linear classifier comprising a first set of variables from a full multivariate dataset, wherein said first linear classifier is capable of answering the classification question with a log odds ratio greater than or equal to a first selected threshold value;
b. removing said first set of variables from the full dataset thereby resulting in a partially depleted dataset;
c. deriving a second linear classifier comprising a second set of variables from the partially depleted dataset, wherein the second linear classifier capable of answering a classification question with a log odds ratio greater than or equal to a second selected threshold value;
d. removing the variables of the second linear classifier from the partially depleted dataset;
e. repeating steps c and d until the second linear classifier generated is not capable of performing with a log odds ratio greater than or equal the first selected threshold value;
wherein the combined set of variables from the derived linear classifiers constitute the necessary set, and the remaining variables in the multivariate dataset constitute the depleted set for answering the classification question.

2. The method of claim 1, further comprising:
g. repeating steps c and d until the second linear classifier generated is not capable of performing with a log odds ratio greater than or equal to a second selected threshold value.

3. The method of claim 2, wherein the first and second selected threshold values are equal.

4. The method of claim 2, wherein the second selected threshold value is less than the first selected threshold value.

5. The method of claim 1, wherein the linear classifiers are generated with an algorithm selected from the group consisting of SPLP, SPLR and SPMPM.

6. The method of claim 1, wherein the multivariate dataset comprises data from polynucleotide array experiments.

7. The method of claim 6, wherein the polynucleotide array experiment comprises compound-treated samples.

8. A set of necessary variables for answering a classification question made according to claim 1.

9. The set of variables of claim 8 wherein the variables are genes.

10. The set of variables of claim 9 wherein the number of genes is 400 or fewer.

11. The set of variables of claim 9 wherein the number of genes is 100 or fewer.

12. An array comprising a set of polynucleotides each representing a gene in the necessary set of claim 8.

13. An array comprising a set of receptors each capable of binding a protein encoded by a gene in the necessary set of claim 8.

14. A subset of genes useful for answering a chemogenomic classification question comprising a percentage of genes randomly selected from a necessary set made according to claim 1, wherein the addition of the genes to the depleted set for the classification question increases the average logodds ratio of the linear classifiers generated by the depleted set.

15. The subset of claim 14, wherein the classification question is selected from those listed in Table 2.

16. The subset of claim 14, wherein the classification question is monoamine re-uptake (SERT) inhibitor and the necessary set consists of the 311 genes listed in Table 5.

17. The subset of claim 16, wherein the randomly selected percentage of genes from the necessary set is 15% and the average logodds ratio is increased to greater than or equal to 3.0.

18. The subset of claim 16, wherein the randomly selected percentage of genes from the necessary set is 26% and the threshold average logodds ratio is to greater than or equal to 4.0.

19. A method for preparing a reagent set comprising:
a. deriving a first linear classifier comprising a first set of genes from a full dataset, wherein said first linear classifier is capable of answering a classification question with a log odds ratio greater than or equal to a first selected threshold value;
b. removing said first set of genes from the full dataset thereby resulting in a partially depleted chemogenomic dataset;
c. deriving a second linear classifier comprising a second set of genes from the partially depleted dataset, wherein the second linear classifier capable of answering a classification question with a log odds ratio greater than or equal to a second selected threshold value;
d. removing said second set of genes from the partially depleted dataset;
e. preparing a plurality of isolated polynucleotides or polypeptides, wherein each polynucleotide or polypeptide is capable of detecting at least one gene of said first and second sets genes.

20. The method of claim 1, further comprising: after step d repeating the steps of (i) deriving a linear classifier; and (ii) removing each additional linear classifier's set of genes from the partially depleted dataset; until the partially depleted dataset is not capable of generating a linear classifier with a log odds ratio greater than or equal to the second selected threshold value.

21. A reagent set for answering a classification question comprising a set of polynucleotides or polypeptides representing a plurality of genes, wherein the addition of a random selection of at least 10% of said plurality of genes to the depleted set for the classification question increases the average logodds ratio of the linear classifiers generated by the depleted set by at least 20%.

22. The reagent set of claim 21, wherein the random selection is of at least 25% of said plurality of genes and the average logodds ratio of the linear classifiers generated by the depleted set by at least 50%.

23. The reagent set of claim 21, wherein the classification question relates to the effect of an in vivo compound treatment on gene expression.

24. The reagent set of claim 21, wherein the classification question is selected from those listed in Table 2.

25. The reagent set of claim 21, wherein the number of genes is 400 or fewer.

26. The reagent set of claim 21, wherein the number of genes is 200 or fewer.

27. An array comprising a set of polynucleotides capable of specifically binding to the reagent set of claim 21.

28. A diagnostic device comprising the reagent set of claim 21.

29. A method of classifying experimental data comprising:
a. providing at least two non-overlapping sufficient sets of variables useful for answering a classification question;
b. querying the experimental data with one of the at least two non-overlapping sufficient sets of variables;
c. querying the experimental data with another of the at least two non-overlapping sufficient sets of variables;
wherein the classification of the data is determined based on the answers to the queries generated by the at least two non-overlapping sets of variables.
